(19) United States
(12) Patent Application Publication
(10) Pub. No.: US 2021/0290593 A1
(43) Pub. Date: Sep. 23, 2021

(54) USE OF SUSTAINED-RELEASE 5-HYDROXYTRYPTOPHAN IN TREATING GASTROINTESTINAL DISORDERS

(71) Applicants: Duke University, Durham, NC (US); The Trustees of Columbia University in the City of New York, New York, NY (US)

(72) Inventors: Jacob P.R. Jacobsen, Durham, NC (US); Marc G. Caron, Hillsborough, NC (US); Michael Gershon, New York, NY (US); Kara Margolis, New York, NY (US)

(21) Appl. No.: 17/250,350

(22) PCT Filed: Jul. 10, 2019
(86) PCT No.: PCT/US2019/041166

§ 371 Date: Jan. 8, 2021

Related U.S. Application Data
(60) Provisional application No. 62/696,396, filed on Jul. 11, 2018.

(51) Int. Cl. A61K 31/405; A61K 45/06; A61K 9/00; A61P 1/10

(52) U.S. Cl. CPC A61K 31/405; A61P 1/10; A61K 9/0053; A61K 45/06

(57) ABSTRACT

The present invention provides, inter alia, methods for treating or ameliorating a gastrointestinal (GI) condition such as constipation in a subject in need thereof, using a sustained release formulation of 5-hydroxytryptophan (5-HTP SR). A kit comprising 5-HTP SR is also provided.

### STATEMENT OF GOVERNMENT SUPPORT

This invention was made with Government Support under DK093786, NS15547, and NIA T35AG044303 awarded by the National Institutes of Health (NIH), and FPR160365 awarded by the Department of Defense (DOD). The U.S. Government has certain rights to this invention.

### FIELD

The present invention provides, inter alia, methods for treating gastrointestinal (GI) conditions using sustained release formulations of 5-hydroxytryptophan (5-HTP SR).

### BACKGROUND

Serotonin (a.k.a. 5-hydroxytryptamine, 5-HT), a neurotransmitter classically known for its roles in sleep and mood, is also critical for central nervous system (CNS) and enteric nervous system (ENS) development, GI motility, and enteric microbiome modulation. Serotonin is synthetized from dietary tryptophan via the immediate precursor 5-hydroxytryptophan (5-HTP) (Turner et al, 2006). The conversion of tryptophan to 5-HTP is mediated by tryptophan hydroxylase (TPH), which is the rate-limiting enzyme responsible for 5-HT synthesis.

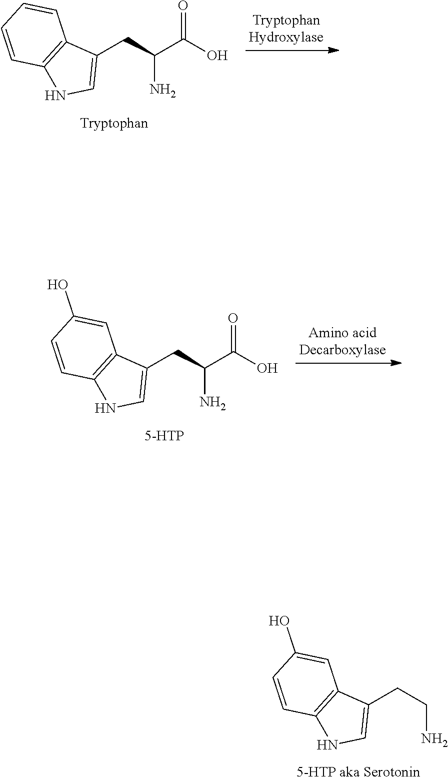 [SMILES: N[C@@H](Cc1c[nH]c2ccccc12)C(=O)O ; N[C@@H](Cc1c[nH]c2ccc(O)cc12)C(=O)O ; NCCc1c[nH]c2ccc(O)cc12]

In the intestine, two different 5-HT depots exist, each with its own rate-limiting isoform of TPH. A large TPH1-dependent 5-HT pool exists in mucosal enterochromaffin (EC) cells, while a smaller TPH2-dependent pool exists in the serotonergic neurons of the ENS. Enteric neuronal 5-HT functions as a neurotransmitter and also a neurogenic growth factor during development and adult life. Both enteric TPH1- and TPH2-derived 5-HT regulate GI motility (Kendig and Grider, 2015). TPH2-derived 5-HT has further been demonstrated to impact intestinal epithelial growth by regulation of crypt epithelial cell proliferation (Gross et al, 2012). Notably, TPH2 is specifically required for CNS-derived 5-HT production (Walther et al, 2003). 5-HTP's pharmacological actions on the gut, therefore, could stem from increased 5-HT production in ENS neurons as well as in non-neural cells, in particular enterochromaffin cells.

On the other hand, administration of exogenous 5-HTP, the immediate precursor of 5-HT (Turner et al, 2006), will bypass both TPH1 and TPH2, and be converted to 5-HT directly, in a reaction catalyzed by the enzyme amino acid decarboxylase. As conversion of 5-HTP to 5-HT is not the rate-limiting factor in 5-HT synthesis, the circulating systemic levels of endogenous 5-HTP (synthesized form tryptophan) are low under baseline conditions, e.g. 0-50 ng/ml plasma (Comai et al, 2010). 5-HTP plasma levels of over 100 ng/ml are typically needed to produce systemic pharmacological effects after administration of exogenous 5-HTP (Gijsman et al, 2002; Veeninga and Westenberg, 1992).

Previous reports teach that slow-release (SR) delivery enhances the therapeutic potential of 5-HTP (U.S. Pat. Nos. 8,969,400; 9,468,627; 7,670,619), i.e. by reducing C_{Max}-related adverse events, by avoiding intermittent sub-therapeutic troughs in 5-HTP systemic exposure, and by reducing dosing to a frequency that is practical in a therapeutic setting. This use of 5-HTP SR concerns methods of treatment that work by delivering exogenous 5-HTP for absorption into the systemic circulation and pharmacological action via 5-HT synthesized by the central nervous system (CNS), from the exogenous 5-HTP having been thus absorbed from the intestine. U.S. Pat. No. 7,670,619 to Mihaylov describes 5-HTP formulations that are comprised of double-layer tablets. One layer contains 5-HTP released rapidly. The other layer contains tryptophan or 5-HTP released progressively over 7 hours or longer. U.S. Pat. No. 9,468,627 to Jacobsen et al. describes various 5-HTP SR delivery approaches that can deliver 5-HTP throughout the GI tract. U.S. Pat. No. 9,468,627 also mentions formulations that mostly are retained in the stomach during 5-HTP drug delivery, i.e. “gastroretentive” 5-HTP SR formulations, to selectively deliver 5-HTP to the upper intestine for absorption into the systemic circulation.

According to the American College of Gastroenterology, constipation is defined as: “a symptom-based disorder defined as unsatisfactory defecation and is characterized by infrequent stools, difficult stool passage, or both.” Over 10% of the population suffers from constipation disorders, with irritable bowel syndrome-constipation dominant and chronic idiopathic constipation being the most prevalent diagnoses (Ford et al, 2014). The pathogenesis of constipation disorders prominently involves (i) reduced intestinal, in particular colonic, motility and (ii) reduced net fluid secretion into the intestine, in particular the colon (Gershon, 2013a; Yang and Ma, 2017). In most cases, the primary cause is not known, i.e. the constipation is idiopathic.

Drugs for constipation (laxatives) broadly fall into five categories: (i) Bulk agents (e.g. psyllium, methylcelluose, calcium polycarbophil, wheat dextrin); (ii) non-absorbed substances (e.g. PEG 3350, lactulose, magnesium salts); (iii) stimulants (e.g. bisacodyl, senna); (iv) secretory drugs (e.g. linaclotide, lubeprostone); and (v) pro-motility drugs (e.g. prucalopride, tegaserod, cisapride). The goal of such agents is to increase intestinal motility and/or increase net secretion of fluids into the intestinal lumen, thereby increasing stool water content and/or facilitating bowel movement. A main pathological locus of constipation is the colon, where fecal matter accumulates due to inadequate evacuation (Wald, 2016). Thus, enhancement of colonic motility and net colonic fluid secretion may be beneficial.

As mentioned, 5-HT is a major regulator of GI function. Specifically, 5-HT promotes GI motility as well as fluid secretion (Borman and Burleigh, 1997; Gershon, 2013a). The 5-HT receptors mediating the pro-motility and pro-secretion effects of 5-HT in the GI are mainly 5-HT₄ receptors and 5-HT₃ receptors, although 5-HT_{2A} receptors, 5-HT_{2B} receptors, and 5-HT₇ receptors may also play a role (Sanger, 2008). Agonists of both 5-HT₄ receptors and 5-HT₃ receptors stimulate GI motility in humans (De Maeyer et al, 2008; Spiller, 2011). Agonists of 5-HT₄ receptors (e.g., cisapride, tegaserod, prucalopride) have won regulatory approval for the treatment of constipation disorders. Selective agonists of the 5-HT₄ receptor alleviate constipation via a pro-motility action. However, off-target toxicity and/or insufficient efficacy are drawbacks for 5-HT₄ receptor agonists in treating constipation disorders (De Maeyer et al, 2008). Selective agonists of the 5-HT₃ receptor also have anti-constipation activity; but, systemic administration of 5-HT₃ receptor agonists are associated with significant nausea (Mawe and Hoffman, 2013). A drug treatment that enabled stimulation of multiple 5-HT receptors simultaneously (5-HT₄ and 5-HT₃ receptors, and possibly also 5-HT_{2A}, 5-HT_{2B}, and 5-HT₇ receptors) would be predicted to provide a more pronounced anti-constipation action.

5-HTP has been studied as an experimental therapeutic in humans, mostly as an antidepressant (Turner et al, 2006). But no regulatorily-approved 5-HTP prescription drugs are currently available, presumably because native 5-HTP, due to rapid pharmacokinetics, is poorly suited as a drug therapy in humans (Jacobsen et al, 2016a). One of the most common peripheral actions associated with 5-HTP administration in humans is accelerated bowel movements and increased fecal water content (Turner et al, 2006). Likewise, in rodents, exogenous 5-HTP administration—even at low doses, <10 mg/kg—is known to be a potent inducer of GI motility and fluid secretion (Wang et al, 2007a). In rodents, the mechanism appears to involve stimulation of both 5-HT₃ and 5-HT₄ receptors (Banner et al, 1996; Pascual et al, 2002). Interestingly, these GI effects occur also at low 5-HTP doses that do not cause any obvious behavioral anomalies (Omori et al, 1973).

There is public mentioning (e.g., on internet health web sites) of 5-HTP as a potential remedy for constipation. However, there are no published peer-reviewed animal or human data directly suggesting 5-HTP can be used to treat constipation or other GI disorders, nor mention of an appropriate dose, dosage form, or other treatment parameters. Likewise, there are no examples or designs of how to make a pharmaceutical 5-HTP dosage form to effectively treat constipation or other GI disorders.

### SUMMARY

Provided herein are methods of treating or ameliorating the effects of a gastrointestinal (GI) condition in a subject in need thereof, comprising administering to the subject an effective amount of a sustained release formulation of 5-hydroxytryptophan (5-HTP SR). In some embodiments, the subject is a mammal, such as humans, veterinary animals, or agricultural animals. In preferred embodiments, the subject is a human.

In some embodiments, the gastrointestinal (GI) condition is constipation. In some embodiments, the GI condition is selected from Irritable Bowel Syndrome (IBS), idiopathic constipation, constipation-predominant Irritable Bowel Syndrome (IBS-C), short gut syndrome, postoperative gut repair, functional visceral pain, visceral hypersensitivity, enteric nervous system (ENS) hypoplasia, deficient late-developing neurons, abnormal enteric epithelial growth and proliferation.

In some embodiments, the GI condition is selected from idiopathic constipation, Irritable Bowel Syndrome (IBS), constipation, constipation-predominant Irritable Bowel Syndrome (IBS-C), opioid-induced constipation, constipation in Parkinson's disease and constipation in autism.

In some embodiments, the GI condition is constipation induced by a drug treatment (e.g., opioid-induced constipation).

In some embodiments, the subject suffers from constipation due to GI deficiency in 5-HT. In some embodiments, the 5-HT deficiency stems from a mutation in the gene encoding tryptophan hydroxylase 2 (TPH2). In some embodiments, the mutation is a R441H or equivalent mutation in the TPH2 gene.

In some embodiments, the administering step is carried out by oral administration.

In some embodiments, the 5-HTP is administered at a rate of about 0.1 g per kg body weight per day. In some embodiments, the 5-HTP SR is administered at a rate of about 0.01 to 0.1 g per kg body weight per day. In some embodiments, the 5-HTP SR is administered at a rate of about 0.001 to 0.01 g per kg body weight per day. In some embodiments, the 5-HTP SR is administered at a rate of about 0.0001 to 0.001 g per kg body weight per day. In some embodiments, the 5-HTP SR is administered at a rate of about 0.00001 to 0.0001 g per kg body weight per day.

In some embodiments, the 5-HTP is orally administered at a dosage of from 5, 10 or 25 mg to 100, 250 or 500 mg; or orally administered at a dosage of from 0.5 to 1 gram.

Also provided is a method for treating visceral pain in a subject in need thereof, comprising administering to the subject an effective amount of 5-HTP SR.

Further provided is a kit for treating or ameliorating the effects of a GI condition in a subject, comprising an effective amount of 5-HTP SR, packaged together with instructions for its use. In some embodiments, the kit further comprises a pro-secretory drug. In some embodiments, the kit further comprises a drug to prevent 5-HT-related nausea. In some embodiments, the kit further comprises an SSRI, or another antidepressant. In some embodiments, the kit further comprises a stimulant. In some embodiments, the kit further comprises an osmotic agent. In some embodiments, the kit further comprises a bulk laxative.

In some embodiments of the foregoing, the 5-HTP SR is a gastroretentive formulation. In some embodiments of the foregoing, the 5-HTP SR is a gastroretentive formulation that further comprises a peripheral decarboxylase inhibitor. In some embodiments of the foregoing, the 5-HTP SR is a gastroretentive formulation that further comprises a peripheral decarboxylase inhibitor at a low dose that does not cause systemically active carbidopa blood levels.

In some embodiments of the foregoing, the 5-HTP is delivered to the upper and lower GI. In some embodiments of the foregoing, the 5-HTP is delivered throughout the intestine.

In some embodiments of the foregoing, the 5-HTP is delivered specifically/selectively to the colon.

Further provided is 5-HTP for use in treating or ameliorating the effects of a gastrointestinal (GI) condition in a subject in need thereof, wherein the medicament comprises a sustained release formulation of 5-hydroxytryptophan (5-HTP SR) as taught herein.

Also provided is the use of 5-HTP in the preparation of a medicament for treating or ameliorating the effects of a gastrointestinal (GI) condition in a subject in need thereof, wherein the medicament comprises a sustained release formulation of 5-hydroxytryptophan (5-HTP SR) as taught herein.

### BRIEF DESCRIPTION OF THE DRAWINGS

FIG. 1. Numbers of total and late-born enteric neurons are decreased in R439H mice. (n=3-4/group). Immunocytochemical detection of ANNA-1 was used as a total neuronal marker; GABA was used as a marker for enteric GABAergic neurons; TH was used as a marker for enteric dopaminergic neurons. (A) Total neurons (ANNA-1+) and GABAergic neurons (GABA+) in the myenteric plexus of ileum. (B) GABAergic neurons as a proportion of total neurons in the myenteric plexus of ileum. (C) Total neurons and dopaminergic neurons (TH+) in the submucosal plexus of ileum. (D) Dopaminergic neurons as a proportion of total neurons in the submucosal plexus of ileum. (E) Total neurons and GABAergic neurons in the myenteric plexus of colon. (F) GABAergic neurons as a proportion of total neurons in the myenteric plexus of colon. (G) Total neurons and dopaminergic neurons in the submucosal plexus of colon. (H) Dopaminergic neurons as a proportion of total neurons in the submucosal plexus of colon. (I-N) Myenteric plexus from ileum of WT (I-K) and R439H (L-N) mice. (I and L) Total neurons (ANNA-1 immunoreactive). (J and M) GABAergic neurons. (K and N) Coincident immunoreactivity between total and GABAergic neurons. (O-T) Submucosal plexus from colon of WT (O-Q) and R439H (R-T) mice. (O and R) Total neurons (ANNA-1 immunoreactive). (P and S) Dopaminergic neurons. (Q and T) Coincident immunoreactivity between total and dopaminergic neurons. Student's unpaired t test was used to compare groups. Data represent the mean±SEM. Scale bars: 25 μm.

FIG. 2. Intestinal motility is slower in R439H than WT mice. (n=12-14/group; 1-3 trials). (A) Total GI transit was measured in vivo after administration of oral carmine red. (B) Colonic motility was estimated by measuring time required to expel a glass bead inserted 2 cm into the rectum. (C) Gastric emptying and (D) small (upper) intestinal transit were measured by fluoroscopy after administration of oral rhodamine dextran. (E and F) Spatiotemporal maps showing CMMCs (arrow) in isolated preparations of colon of WT (E) and R439H (F) mice (n=8-10/group). The ordinate represents time, and the abscissa represents oral-to-anal distance. The width of the gut (mm), indicative of contractions, was pseudocolored. (G) CMMC frequency and (H) CMMC velocity were measured in MATLAB 2013b after construction of spatiotemporal maps from video imaging. Student's t test was used to compare groups. Data represent the mean±SEM.

FIG. 3. Immediate-release 5-HTP (5-HTP IR) modulates in vivo motility and in vitro peristaltic contractions. (n=6-10/group). (A) Total GI transit was measured after administration of oral carmine red in control mice and mice exposed to 30 and 100 mg/kg of intraperitoneal 5-HTP IR. (B) Colonic motility was estimated by measuring time required to expel a glass bead inserted 2 cm into the rectum in control mice and mice exposed to 30 and 100 mg/kg of intraperitoneal 5-HTP IR. (C) CMMC frequency and (D) CMMC velocity in consecutive measurements of isolated colon before and after receiving a single extraluminal dose of 1 μM 5-HTP IR (n=3/group). (E) CMMC frequency and (F) CMMC velocity in consecutive measurements of isolated colon before and after receiving an intraluminal dose of 1 μM 5-HTP IR (n=4/group). (G-J) Spatiotemporal maps showing CMMCs (white arrow) in isolated preparations of colon in WT (G) and R439H (I) mice before administration of intraluminal 5-HTP IR, and WT (H) and R439H (J) mice after administration of intraluminal 5-HTP IR. Student's unpaired t test and one-way ANOVA were used to compare groups, respectively, to compare single and multiple means. Data represent the mean±SEM. Ctrl, Control.

FIG. 4. Administration of sustained-release 5-HTP during adulthood rescues mice from the ENS abnormalities associated with the R439H mutation. (n=3-4/group). Immunocytochemical detection of ANNA-1 was used as a total neuronal marker; GABA was used as a marker for enteric GABAergic neurons; TH was used as a marker for enteric dopaminergic neurons. (A) Total neurons (ANNA-1+) and (B) GABAergic neurons (GABA+) in the myenteric plexus of ileum. (C) GABAergic neurons as a proportion of total neurons. (D) Total neurons and (E) dopaminergic neurons (TH+) in the submucosal plexus of ileum. (F) Dopaminergic neurons as a proportion of total neurons. (G-R) Myenteric plexus from ileum of (G-I) WT, (J-L) WT treated with 5-HTP SR, (M-O) R439H, and (P-R) R439H treated with 5-HTP SR. (G, J, M, P) Total neurons (ANNA-1 immunoreactive). (H, K, N, Q) GABAergic neurons. (I, L, O, R) Coincident immunoreactivity between total and GABAergic neurons. One-way ANOVA and Fisher's LSD test was used to compare groups. Data represent the mean±SEM. Scale bars: 25 μm. Veh, Vehicle.

FIG. 5. Administration of sustained-release 5-HTP during adulthood reverses abnormalities in motility associated with the R439H mutation. (n=10-14/group, 1-3 trials). (A) Total GI transit was measured in vivo after administration of oral carmine red. (B) Colonic motility was estimated by measuring time required to expel a glass bead inserted 2 cm into the rectum. (C) Gastric emptying and (D) small (upper) intestinal transit were measured by fluoroscopy after administration of oral rhodamine dextran. (E) CMMC frequency and (F) CMMC velocity were measured in MATLAB 2013b after construction of spatiotemporal maps from video imaging (n=5-9/group). (G-J) Spatiotemporal maps showing CMMCs (arrow) in isolated preparations of colon of WT (G) and R439H (H) mice receiving control chow, and WT (H) and R439H (J) mice receiving 5-HTP SR. The ordinate represents time, and the abscissa represents oral-to-anal distance. The width of the gut (mm), indicative of contractions, was pseudocolored. One-way ANOVA and Fisher's LSD test was used to compare groups. Data represent the mean±SEM. Veh, Vehicle.

FIG. 6. The R439H mutation leads to abnormal parameters of intestinal epithelial homeostasis that are ameliorated by administration of 5-HTP SR. (n=4-6/group) (A) Villus height was measured as distance from base of villus to tip (30/mouse). (B) Crypt perimeter was measured by tracing the border of each crypt up to the base of each villus (30/mouse). (C-F) Sections of ileum stained with hematoxylin and eosin showing an individual villus and neighboring crypts in WT (C), WT with 5-HTP (D), R439H (E) and R439H with 5-HTP (F) mice. (G) Counts of enterochromaffin cells (EC; 5-HT+), as a proportion of individual villus area, in ileum. (H) Counts of enteroendocrine cells (EE; chromogranin-A+), as a proportion of individual villus area, in ileum. (I-L) Sections of ileum stained with bisbenzimide (DNA; blue) and 5-HT (EC cell; red) in WT (I), WT with 5-HTP (J), R439H (K) and R439H with 5-HTP (L) mice. (M) Transcripts of TPH2 mRNA in ileum as a proportion of GAPDH (n=14-21/group). (N) Transcripts of SERT mRNA in ileum as a proportion of GAPDH (n=14-21/group). 1-way ANOVA and Fisher's LSD test was used to compare groups. Data represents the mean±SEM. Scale bars: 25 μm. Veh, Vehicle.

FIG. 7. Microbiome characterization revealed key differences between R439H mice and WT mice that resolved with 5-HTP SR treatment. (A) Family-level analysis identified decreases in several families in the R439H mice compared to WT mice. Features: A, Anaeroplasmataceae; B1, Bacteroidaceae; B2 Bifidobacteriaceae; C1, Clostridiaceae_1; C2, Coriobacteriaceae; D1, Deferribacteriaceae; D2, Desulfovibrionaceae; E1, Enterobacteriaceae; E2, Erysipelotrichaceae; E3, Eubacteriaceae; H, Helicobacteraceae; L1, Lachnospiraceae; L2, Lactobacillaceae; M, Mycoplasmataceae; P1, Pasteurellaceae; P2, Peptostreptococcaceae; P3, Porphyromonadaceae; P4, Prevotellaceae; R1, Rikenellaceae; R2, Ruminococcaceae; S, Sutterellaceae; UA, Unclassified Alphaproteobacteria; UB1, Unclassified Bacteria; UB2, Unclassified Bacteroidales; UB3, Unclassified Bacteroidetes; UB4, Unclassified Betaproteobacteria; UC1, Unclassified Candidatus Saccharibacteria; UC2, Unclassified Clostridia; UC3, Unclassified Clostridiales; UD, Unclassified Deltaproteobacteria; UF, Unclassified Firmicutes; UP, Unclassified Proteobacteria; V, Verrucomicrobiaceae. (B) Several OTUs normalized to WT in the R439H mice following 5-HTP SR treatment, including increases in Porphyromonadaceae, Lachnospiraceae, and Akkermansia, with decreases in Anaeroplasma and Clostridiales. (C) Porphyromonadaceae was increased in mice with slower colonic motility. (D) Increased Akkermansia was associated with slower total GI transit time.

FIG. 8. Morphine-induced constipation and reversal by 5-HTP in mice. Morphine 10 mg/kg induced constipation, as determined by slowed colonic motility. 5-HTP 30 mg/kg reversed the morphine-induced constipation and by itself enhanced colonic motility. *, p<0.05, group comparisons as indicated.

FIG. 9. Colonic motility. Effect of prucalopride vs 5-HTP. 5-HTP is more potent than prucalopride at lower doses *, p<0.05, compared to control. #, p<0.05, prucalopride vs 5-HTP.

FIG. 10. Is a plot of plasma concentration of 5-HTP against time for oral, colonic, and intravenous (IV) administration of 5-HTP in human volunteers. Average baseline 5-HTP plasma levels prior to 5-HTP administration for oral, intra-colonic, and I.V. sub-experiments were 6.7, 4.0, and 2.8 ng/ml, respectively. Oral 5-HTP 200 mg had a bioavailability of F=20% and produced maximal average 5-HTP plasma (C_{Max}) levels of 370 ng/ml. Intra-colonic 5-HTP 200 mg had a bioavailability of F=4% and produced maximal average 5-HTP plasma (C_{Max}) levels of 47 ng/ml.

FIG. 11. (A) Diagram of example embodiment of colon-selective 5-HTP SR solid dosage form. In this embodiment the dosage form consists of two layers, a coating and a slow-release core. The coating ensures that no or minimal 5-HTP is delivered to the upper intestine, by isolating the slow-release core from the aqueous phase in the intestine, i.e. the chyme and fecal matter. The slow-release core delivers the 5-HTP over a prolonged period, thereby causing sustained pharmacological action while reducing adverse events by lowering local tissue C_{Max} 5-HTP values. Coating: The coating can be pH-dependent, only dissolving at pH˜7, as occurs in the terminal ileum; can be time-dependent, e.g. slowly dissolving over 3-5 h, the typical transit time from stomach to colon; can be dependent on digestion by colonic microbiota; or can utilize a combination of the approaches. Slow-release core: The slow-release core can be realized using available matrix, osmotic, soft-gel, erosion, etc. technologies. (B) Schematic of the drug delivery functioning of the example embodiment shown in (A). The coating remains essentially intact in the stomach, jejunum, and proximal ileum, preventing substantial 5-HTP delivery. Only in the terminal ileum, the ileocecal junction, cecum, or in the colon will the coating disintegrate, triggering commencement of 5-HTP delivery throughout the colon. (C) Schematic of the anti-constipation pharmacological effect of colon-selective 5-HTP SR in the colon. In the constipated state, dry and hard fecal matter accumulates in the colon, causing pain, bloatedness, and general discomfort. 5-HTP delivered is taken up by enterochromaffin and potentially other epithelial and non-epithelian cells (e.g., neurons) and converted into 5-HT. The 5-HT is released and acts on 5-HT₃, 5-HT₄ and other 5-HT receptors located on cells involved in smooth muscle actin and fluid secretion, to potently enhance both colonic motility and new fluid secretion into the colonic lumen. The fluid secretion softens the hard stool, which facilitates the evacuation of the of the stool by the enhanced colonic motility. Thus, the enhanced colonic motility and fluid secretion synergizes in resolving constipation.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including”, “comprising,” or “having,” and variations thereof, is meant to encompass the elements listed thereafter and equivalents thereof as well as additional elements. Embodiments recited as “including”, “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

Unless otherwise defined, all technical terms used herein have the same meaning as commonly understood by one of ordinary skill in the art to which this disclosure belongs.

There has been no description in the art heretofore of methods, formulations, or other details of how to use 5-HTP to effectively treat GI disorders. In contrast to existing 5-HT-stimulating drugs for constipation, i.e. 5-HT₄ receptor agonists (e.g. Motegrity® (prucalopride)), 5-HTP can activate multiple 5-HT receptors involved in GI motility. Further, 5-HTP is also a powerful inducer of fluid secretion into the intestinal lumen. By itself, increased fluid secretion into the intestinal is well-established to exert an anti-constipation effect (e.g. the FDA-approved pro-secretory drugs Linzess® (linaclotide) and Amitiza® (lubiprostone)). Hence, a 5-HTP-based anti-constipation medication is predicted to be more effective than these existing drugs, by stimulating motility more potently than 5-HT₄ receptor agonists while simultaneously increasing fluid secretion.

In the present invention, it is also shown that the abnormalities in TPH2-mediated 5-HT production in a subject with certain mutation (e.g., R441H in human) in the gene encoding TPH2 could cause the abnormalities in GI function that result from 5-HT deficiency in the ENS. It is further shown that these abnormalities can be treated through mechanism-guided intervention with 5-HTP SR. It is further shown that 5-HTP can act as a general agent to enhance motility and secretion, which is predicted to counter-act constipation arising from many primary causes, related to anomalies in 5-HT, and otherwise.

Accordingly, one embodiment of the present invention is a method of treating or ameliorating the effects of a gastrointestinal (GI) condition in a subject. This method comprises administering to the subject an effective amount of a sustained release formulation of 5-hydroxytryptophan (5-HTP SR).

In some embodiments, the subject is a mammal that can be selected from the group consisting of humans, veterinary animals, and agricultural animals. Preferably, the subject is a human.

As used herein, “gastrointestinal diseases”, “gastrointestinal conditions” or “GI conditions” refer to diseases or conditions involving the gastrointestinal tract. Non-limiting examples of gastrointestinal conditions include Irritable Bowel Syndrome (IBS), idiopathic constipation, constipation-predominant Irritable Bowel Syndrome (IBS-C), short gut syndrome, postoperative gut repair, functional visceral pain, visceral hypersensitivity, enteric nervous system (ENS) hypoplasia, deficient late-developing neurons, abnormal enteric epithelial growth and proliferation. In certain embodiments, the GI condition is constipation-predominant Irritable Bowel Syndrome (IBS-C). In some embodiments, the GI condition is drug-induced such as opioid-induced constipation.

In some embodiments, the GI condition is constipation related to Parkinson's disease. In some embodiments, the GI condition is constipation related to psychiatric illness. In some embodiments the psychiatric illness is depression. In some embodiments the psychiatric illness is autism.

Other GI disorders that 5-HTP SR may treat include those caused by decreased intestinal surface area or dysfunctional enteric mucosal proliferation. Examples of such GI disorders include, but are not limited to, short bowel syndrome, and postoperative gut repair.

In some embodiments, the subject has GI 5-HT deficiency. In some embodiments, the subject has a mutation in a gene encoding tryptophan hydroxylase 2 (TPH2). In certain embodiments, the mutation is R441H in human, or other equivalent mutations in other nonhuman subjects.

In some embodiments, the subject has normal 5-HT function in the GI, and the 5-HTP SR therapy and resultant enhancement of 5-HT function compensates for a primary pathogenic anomaly unrelated to 5-HT.

As used herein, a “sustained release” or “slow release” formulation of 5-HTP (5-HTP “SR”) refers to a formulation with the ability to release 5-HTP at a slow rate, such that the plasma T_{1/2} is delayed/extended and/or T_{Max} is decreased as compared to an immediate release formulation, or plasma T_{Max} is delayed and/or C_{Max} is decreased as compared to an immediate release formulation, while the duration of therapeutically active 5-HTP exposure is prolonged (i.e., T_{1/2} is extended). The positive consequences are better clinical effectiveness, better safety, and higher convenience for the patient. The terms “5-HTP sustained release”, “5-HTP at a slow rate”, and “5-HTP at a slow release” and “5-HTP slow-release/5-HTP SR” are used interchangeably and refer to the ability to cause the 5-HTP to be released in the subject at a slower rate than if administered directly, i.e. as an immediate-release form, such as a tablet, solution, or powder.

Slow-release (SR) formulations 5-HTP may be prepared by formulation methods known in the art, such as in the latest edition of Advances in Delivery Science and Technology (Springer; 2011 edition) and Modified-Release Drug Delivery Technology (Drugs and the Pharmaceutical Sciences Book 184; 2nd Edition). Non-limiting examples of SR formulations include lipophilic matrix, hydrophilic matrix, mixed lipophilic and hydrophilic matrix, osmotic, erosion, diffusion, soft-gel, microparticle, micro-tablet, and capsule systems. Appropriate excipients such as binders, glidants, lubricants, fillers, anti-oxidants, disintegrants, coloring agents, and coatings can be included as appropriate.

To achieve colon-selective delivery of 5-HTP, the dosage form may include a coating that prevents or inhibits delivery to occur before said coating dissolves and the active 5-HTP core is exposed. The active 5-HTP core can be uniform or comprise two or more layers or sub-compartment with different 5-HTP delivery characteristics. Upon exposure, contact with water in the GI triggers commencement of drug-delivery of 5-HTP. Non-limiting examples of coatings conferring colon-selectivity includes pH-dependent coatings, time-dependent coatings, and microbiota-dependent coatings, as reviewed in Amidon (Amidon et al, 2015, Colon-targeted oral drug delivery systems: design trends and approaches. AAPS Pharm SciTech 16(4): 731-741). In some cases, a combination of coatings, optionally using different principles, e.g. both pH and time-dependent, can be used.

pH-dependent coatings may be comprised of methacrylate, derivatives of methacrylic acid, such as Eudragit® S-100; Eudragit® S 12.5; Eudragit® FS 30 D; and Eudragit® FS 100. Alternatively, certain poly-saccharides can be used. These pH-sensitive polymers dissolve at pH >/=7, which is usually only encountered in the terminal ileum. The 5-HTP containing core is thus only exposed in the terminal ileum, cecum, or colon, and the compound is pre-dominantly or only delivered to the colon.

Time-dependent coatings erode at a pre-specified rate, e.g. 4-6 h, exposing the 5-HTP core. As upper intestine transit time is usually 3-4 h, the delivery will usually commence in the colon. Non-limiting examples of polymers that will erode at a predictable rate, allowing for time-dependent delivery, includes waxes, hydroxypropylmethylcellulose and Eudragit RS100.

Microbiota-dependent coatings are comprised of material, usually polymers, usually polysaccharides, that are degraded by enzymes secreted by the colonic microbiota, but not by enzymes secreted by the upper GI system, e.g. the pancreas. Non-limiting examples of such polymers include ethylcellulose and glassy amylose.

In some embodiments the delivery profile is substantially linear, or zero order. In some embodiments the delivery profile is hyperbolic, or 1^{st} order. In some embodiments, delivery onset exhibits a lag upon exposure to water in the GI. The 5-HTP delivery duration will typically be about 4 hours to about 24 hours; in some embodiments about 12 hours; in other embodiments about 4 hours to about 6 hours; in some embodiments about 6 hours to about 12 hours. In some embodiments a short delivery profile will be desired, such as about 1 hour to about 4 hours. The delivery profile can be established by dissolution testing according to standard methodology, as specified by the United States Pharmacopeia.

In some embodiments, the 5-HTP SR is administered to the subject by oral administration at a rate of about 0.1 g per kg body weight per day. This dose is based on the findings described herein that about 1 g per kg body weight per day in mice will counter-act the constipation phenotype in ENS 5-HT deficient mice. Generally, the interspecies scaling factor between mice and humans are in the range of 1/10, meaning that it is predicted that humans will need 1/10th the per kg dose of a drug to produce the same systemic exposure and pharmacological effect.

In some embodiments, the 5-HTP SR is administered at a rate of about 0.01 to 0.1 g per kg body weight per day. In some embodiments, the 5-HTP SR is administered at a rate of about 0.001 to 0.01 g per kg body weight per day. In some embodiments, the 5-HTP SR is administered at a rate of about 0.0001 to 0.001 g per kg body weight per day. In some embodiments, the 5-HTP SR is administered at a rate of about 0.00001 to 0.0001 g per kg body weight per day.

In some embodiments, the administered dose of 5-HTP may be 5, 10 or 25 mg to 100, 200, 300 or 500 mg per oral dosage form. In some embodiments, the dose may be 100 mg to 250 mg. In some embodiments, the dose may be 10 mg to 100 mg. In some embodiments the dose may be 250 mg to 500 mg. In some embodiments, the dose may be higher, such as 500 mg to 1000 mg.

A further embodiment of the present invention is a method of treating visceral pain in a subject. This method comprises administering to the subject an effective amount of a 5-HTP SR formulation. In some embodiments, 5-HTP SR may treat visceral hypersensitivity, manifest as visceral pain, which is used for a diagnosis of irritable bowel syndrome. Low 5-HT levels have been found in some patients with constipation predominant IBS (IBS-C) (Gershon, 2013b). One of the ways in which 5-HT agonists have been able to increase intestinal motility and decrease pain is by activation of the 5-HT₄ receptor. In the intestine, the binding of 5-HT to the 5-HT₄ results in an increase of gastrointestinal motility (Sengupta et al, 2014). In the brain, the activation of 5-HT₄ receptors has been shown to decrease visceral hypersensitivity (Farzaei et al, 2016). A 5-HTP SR drug, depending on design, by increasing the available 5-HT in the brain and intestine, could therefore not only increase GI motility and secretion, but also ameliorate visceral pain.

Another embodiment of the present invention is a kit for treating or ameliorating the effects of a gastrointestinal (GI) condition or visceral pain in a subject. This kit comprises an effective amount of a sustained release formulation of 5-hydroxytryptophan (5-HTP SR), a second agent to treat the GI condition, optionally packaged together with instructions for its use.

It is also shown in the findings presented herein that 5-HTP potently stimulates total gastric motility and colonic motility under conditions where 5-HT deficiency per se is not present. Therefore, dosage forms of 5-HTP are provided that will stimulate intestinal motility and thereby counteract and treat constipation, irrespective of the primary cause.

Several 5-HTP SR dosage forms with targeted delivery to certain portions of the GI tract are described, which may be used to treat constipation and GI disorders using variant mechanisms, non-limiting examples of which are further provided below.

### Example SR Formulation 1: 5-HTP Gastroretentive Formulation

The 5-HTP gastroretentive dosage form will remain in the stomach and deliver 5-HTP for absorption by the upper intestine. From here 5-HTP will be carried by the blood stream to neurons of the enteric nervous system neurons and enterochromaffin cells of the jejunum, ileum, colon, and rectum (neurons only). These cells will convert 5-HTP to 5-HT, which, upon extracellular release, will enhance GI motility and secretion, and thereby alleviate constipation. Simultaneously, a substantial proportion, i.e. the majority, of the administered 5-HTP will enter directly from the GI lumen (mostly in the jejunum), into the enterochromaffin cells, and likely other cell types, wherefrom it is converted into 5-HT, which will act locally to enhance GI motility and secretion in the upper intestine. Thus, 5-HTP will be delivered via the blood stream to neurons, enterochromaffin cells, and other cells, along the entire GI tract and simultaneously absorbed directly from the lumen by predominantly enterochromaffin cells and other cells of the GI epithelium. 5-HTP will be converted to 5-HT along the entire GI tract.

Various drug delivery systems exist which will be appropriate to produce the desired gastroretentive delivery, including, but not limited to, swelling, buoyant, particulate, magnetic, and mucoadhesive systems. See, e.g., Lopes et al, 2016.

This type of formulation may provide broad stimulation of 5-HT function in the ENS and enterochromaffin cells along the entire GI tract, including the stomach. Thus, this formulation may be particularly beneficial in treating conditions where deficient motility and secretion is present and pathogenic throughout the GI tract. Further, by also enhancing 5-HT signaling in the CNS, central antinociceptive pathways may be engaged.

This type of formulation may have particular relevance for constipation accompanied by mood disorders, such as depression and anxiety. Further, this formulation may have particular relevance for GI disorders where impaired gastric function is involved, including, but not limited to, gastroparesis. Moreover, this type of formulation may be advantageous in patients with comorbid disorders treated with levodopa/carbidopa, including but not limited to Parkinson's disease.

### Example SR Formulation 2: 5-HTP+a Peripheral Decarboxylase Gastroretentive Formulation

This gastroretentive dosage form will remain in the stomach and deliver 5-HTP+a peripheral decarboxylase inhibitor (PDI) for absorption by the upper intestine. A PDI, when present with 5-HTP, will inhibit or decrease conversion of 5-HTP to 5-HT. Examples of PDIs include, but are not limited to, carbidopa and benserazide. From the upper intestine, the 5-HTP will be carried by the blood stream to neurons of the enteric nervous system neurons and enterochromaffin cells of the jejunum, ileum, colon, and rectum (neurons only). In some embodiments, the dose of PDI will be so low as to act only locally in the upper intestine to protect 5-HTP against conversion to 5-HT, without causing systemic pharmacologically active PDI levels, i.e. PDI plasma levels less than 40 ng/ml on average during therapy. Such low PDI doses will typically be in the range of 5-50 mg per day. Thus, while 5-HTP will be delivered via the blood stream to neurons, enterochromaffin cells, and other cells, along the entire GI tract. 5-HTP will be converted to 5-HT predominantly in the lower part of the upper GI (i.e., the ileum), the colon, and the rectum.

As noted above, various drug delivery systems exist which will be appropriate to produce the desired gastroretentive delivery, including, but not limited to, swelling, buoyant, particulate, magnetic, and mucoadhesive systems. See, e.g., Lopes et al, 2016.

This type of formulation may provide broad stimulation of 5-HT function in the ENS and enterochromaffin cells predominantly in the ileum, colon, and rectum. Thus, this formulation may be particularly beneficial in treating conditions where deficient motility and secretion is present and pathogenic in the ileum, colon, and rectum. Further, by also enhancing 5-HT signaling in the CNS central nociceptive pathways may be engaged. Moreover, the inclusion of a PDI may enhance 5-HTP bioavailability, which enables a smaller solid dosage form size and facilitates a higher possible 5-HTP plasma level and a stronger pharmacological effect.

This type of formulation may have particular relevance for constipation accompanied by mood disorders, such as depression and anxiety. Further, this type of formulation may have particular relevance in subjects with gastric or upper intestinal sensitivity to 5-HTP, a phenomenon known to occur in some patients (van Hiele, 1980, PMID:6967194).

### Example SR Formulation 3: Intestinal Delivery of 5-HTP

The intestinal delivery dosage form delivers 5-HTP along the three main segments—jejunum, ileum, colon—of the GI tract. In this instance, the dosage form is not retained in the stomach, and transits freely into the intestine. The dosage form will travel down the intestine by mass movement, while delivering 5-HTP. The 5-HTP will be taken up from the lumen, by enterochromaffin cells, and other cells, and most will be converted into 5-HT, which, when released extracellularly, will enhance GI motility and secretion. Less 5-HTP will be delivered to ENS neurons via the blood stream. In some embodiments, the 5-HTP delivery rate is essentially constant from the stomach through the colon. In some embodiments, the 5-HTP delivery is restricted to the intestine—to avoid 5-HT stimulation in the stomach, as this may cause emesis (van Hiele, 1980)—which can be achieved using pH-sensitive enteric coating. In some embodiments, the 5-HTP delivery rate is essentially constant from the stomach through the colon. In some embodiments, the 5-HTP delivery is restricted to the intestine. In some embodiments, the 5-HTP delivery rate is restricted to the intestine and is essentially constant throughout the intestine. In some embodiments, the 5-HTP delivery rate is higher once the dosage form reaches the colon. In some embodiments, approximately 20%, 30%, 40%, 50%, 60%, 70%, 80%, or 90% of the 5-HTP is delivered to the colon vs. the jejunum and ileum combined. In some embodiments, the 5-HTP is delivered such that systemic plasma 5-HTP levels are below 100 ng/ml, which will avoid significant systemic and/or CNS pharmacological action of 5-HTP.

Various drug delivery systems exist which will be appropriate to produce the desired intraluminal delivery profile, including, but not limited to, matrix, particulate, osmotic, erosion, gels, dual-layer, biodegradable, pH-sensitive, and timed-release systems. See, e.g., Park, 2014.

This type of formulation may provide selective augmentation of 5-HT function in the GI without, or with less, effect on the systemic periphery or CNS. This will be advantageous, as 5-HTP adverse effects related to systemic peripheral and CNS exposure will be minimized, and the potential unwanted interactions with concomitant 5-HTergic drug treatment (e.g., with SSRIs, other antidepressants, triptans) will be reduced. Further, for a given tablet 5-HTP dose strength local concentrations of 5-HTP, and hence 5-HT, will be higher in the GI segments below the jejunum, which can yield a stronger pharmacological effect.

This type of formulation may have particular relevance for constipation arising from multiple primary causes, including, but not limited to, IBS-C, opioid constipation, constipation in autism, constipation caused by to medical therapies. Further, this type of formulation may have particular relevance for constipation therapy when 5-HTP could potentially interact with other drug therapy, including, but not limited to, psychiatric, neurological, and analgesic therapy. Moreover, this type of formulation may have particular relevance for GI disorders affecting the jejenum or ileum, such as postoperative gut repair, short bowel syndrome, small intestine dysmotility, small intestine pseudo-obstruction.

### Example SR Formulation 4: Colonic Delivery of 5-HTP

The colonic delivery dosage form delivers 5-HTP (substantially all or the majority of the 5-HTP dose) only to the colon. In this instance, the dosage form travels through the stomach and upper GI and only starts to deliver 5-HTP when reaching the colon, or just before, in the terminal ileum or cecum. The 5-HTP will be taken up from the lumen, by enterochromaffin cells, and other cells, and most 5-HTP will be converted into 5-HT, which, when release extracellularly, will enhance GI motility and secretion. In some embodiments, the 5-HTP delivery rate is essentially constant. In some embodiments, the systemic plasma 5-HTP levels are below 100 ng/ml, which will avoid significant systemic or CNS pharmacological action of 5-HTP.

The 5-HTP dose can be lower, as compared to systemic 5-HTP delivery, in some embodiments 1 g per day or less, in other embodiments less than 0.5 g per day, and in still other embodiments less than 0.25 g per day. In some embodiments, the administered dose of 5-HTP may be 5, 10 or 25 mg to 100, 200, 300 or 500 mg per day. In some embodiments, the dose may be 100 mg to 250 mg per day. In some embodiments, the dose may be 10 mg to 100 mg per day. In some embodiments the dose may be 250 mg to 500 mg per day. In some embodiments, the dose may be higher, such as 500 mg to 1000 mg per day.

As also noted above, various drug delivery systems exist which will be appropriate to produce the desired specific colonic intraluminal delivery profile, including, but not limited to, timed-release, PH-sensitive, microparticulate, pro-drugs, osmotic, and biodegradable systems. See, e.g., Amidon et al, 2015; Dar et al, 2017; Dhaneshwar, 2014 (PMID:28277824; PMID:24707139; PMID:17725524).

“Colon selective” or “colon specific” as used interchangeably herein and denotes a drug delivery approach where drug delivery is either absent or substantially curtailed until the dosage form arrives in the colon. Colon selective drugs are mostly used for inflammatory and infectious diseases localized to the colon. The approaches most commonly used for colon selective drug delivery can be generally divided into (i) approaches involving pH-dependent coating polymers, (ii) time-dependent approaches, and (iii) polysaccharides degraded by colonic microbiota, and (iv) combined approaches (e.g. pH-responsive+time-delayed). Table 1 provides non-exhaustive examples of FDA-approved/late-clinical stage oral solid dosage form drugs using colon selective formulation approaches.

TABLE 1
Colon Selective Oral Solid Dosage Form Drugs

| Compound | Status | Indication | Formulation | Approach |
|---|---|---|---|---|
| Beclomethasone | FDA-approved | IBD | Eudragit-L 100-55 | pH-dependent |
| (Clipper ®) | | | coated tablet | |
| Mesalamine | FDA-approved | Ulcerative | Matrix tablet with | pH-dependent + time-delayed |
| (Lialda ®) | | Colitis | Eudragit-coating | |
| Mesalamine | FDA-approved | IBD | Microbeads with | Time-delayed |
| (Pentasa ®) | | | Ethylcellulose | |
| Parnaparin | Phase III | IBD | Matrix tablet with | pH-dependent |
| | | | Eudragit-coating | |
| Budesonide | FDA-approved | IBD | Microbeads with | pH-dependent + time-delayed |
| (Entocort EC ®) | | | Eudragit-coating | |
| Budesonide | FDA-approved | IBD | Matrix tablet with | pH-dependent + time-delayed |
| (Uceris ®) | | | Eudragit-coating | |
| Rifamycin | FDA-approved | Travelers | Matrix tablet with | pH-dependent + time-delayed |
| (Aemcolo ®) | | Disease | Eudragit-coating | |

Colon selectivity is most commonly secured by pH-dependent coating of the solid dosage form with a coating that only dissolves in the colon or in the terminal ileum. Using these coatings takes advantage of the fact that the pH gradually increases in the upper intestine from about pH=6 to about pH=7.4 in the terminal ileum (Fallingborg, 1999). Methacrylate-based polymers such as the commercially available Eudragit S-100 reliably dissolve at pH >7. Coating a tablet, capsule, or other solid dosage form with Eudragit S-100 shields the dosage form from the aqueous phase (chyme, water, fecal matter) in the intestine. Upon arriving in the terminal ileum, where the pH usually rises above 7, the Eudragit S-100 coating disintegrates. This exposes the core of the dosage form loaded with the active compound to the aqueous phase, which causes drug delivery to commence.

One preferred embodiment of colon specific delivery of 5-HTP uses the combined time-dependent/pH-dependent lipophilic/hydrophilic system as described in U.S. Pat. No. 7,410,651 for the colon selective delivery of budesonide and as described in U.S. Pat. No. 8,263,120 for the colon selective delivery of rifamicin.

The colon specific formulation may provide selective augmentation of 5-HT function in the colon without affecting, or only minimally affecting, the systemic periphery or CNS. This will be advantageous, as 5-HTP adverse effects related to systemic peripheral and CNS exposure will be minimized, and the potential unwanted interactions with concomitant 5-HTergic drug treatment (e.g. with SSRIs, other antidepressants, triptans) will be minimized. Further, safety may be improved as adverse events, e.g. nausea and emesis, related to stimulation of 5-HT receptors in the upper GI will be reduced or minimized. Moreover, for a given tablet 5-HTP dose strength local concentrations of 5-HTP, and hence 5-HT, will be higher in the colon, which can yield a stronger pharmacological effect.

The colon specific formulation is tailored to GI disorders where the primary pathogenic locus is the colon, and may have particular relevance for constipation arising from multiple primary causes, including, but not limited to, IBS-C, opioid constipation, constipation in autism, constipation caused by other medical therapies. Further, this type of formulation may have particular relevance for constipation therapy when 5-HTP could potentially interact with other drug therapy, including, but not limited to, psychiatric, neurological, and analgesic therapy. Moreover, this type of formulation may have particular relevance for GI disorders primarily affecting the colon, such as primary constipation and idiopathic constipation.

The present invention is further described in the following non-limiting examples.

### EXAMPLES

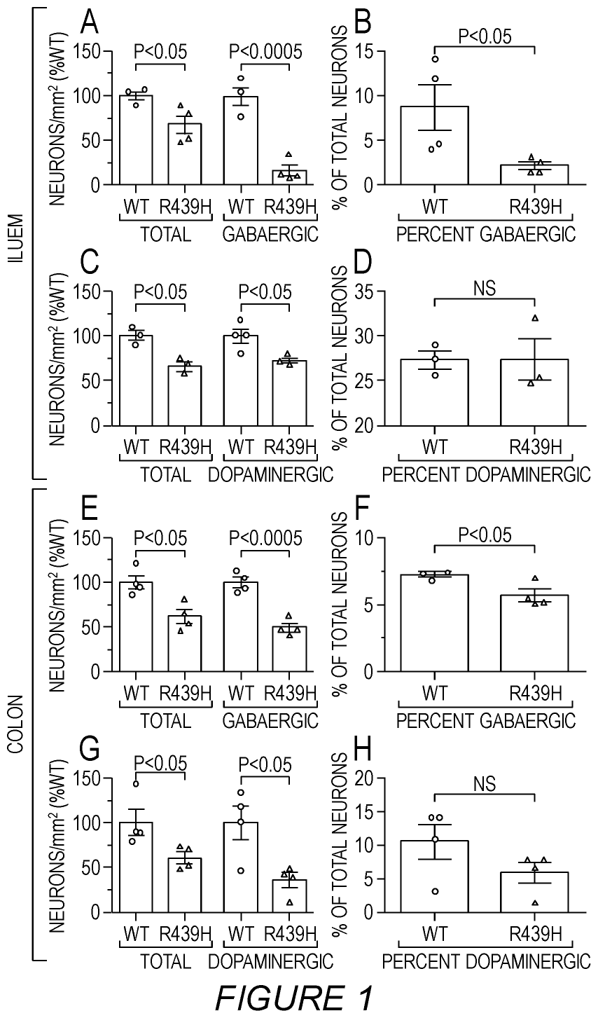

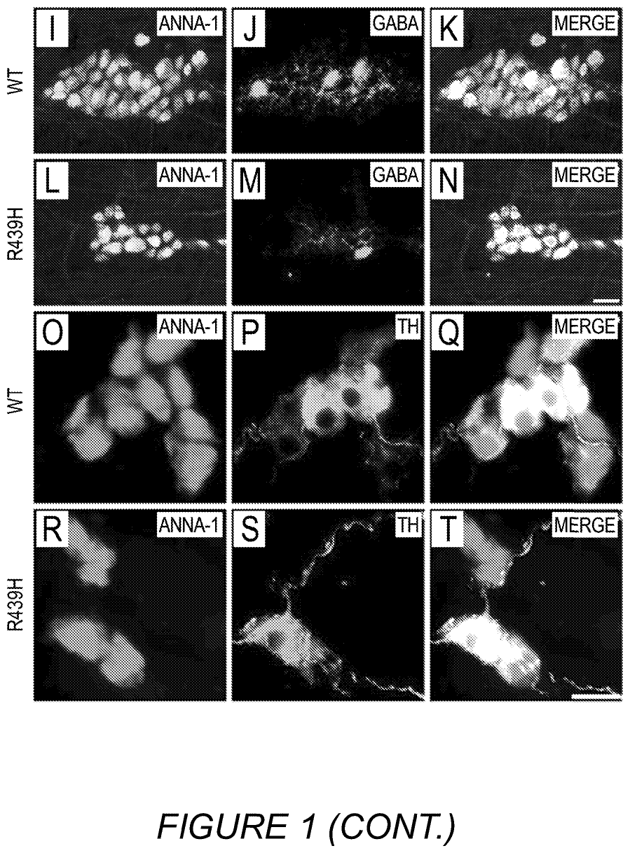

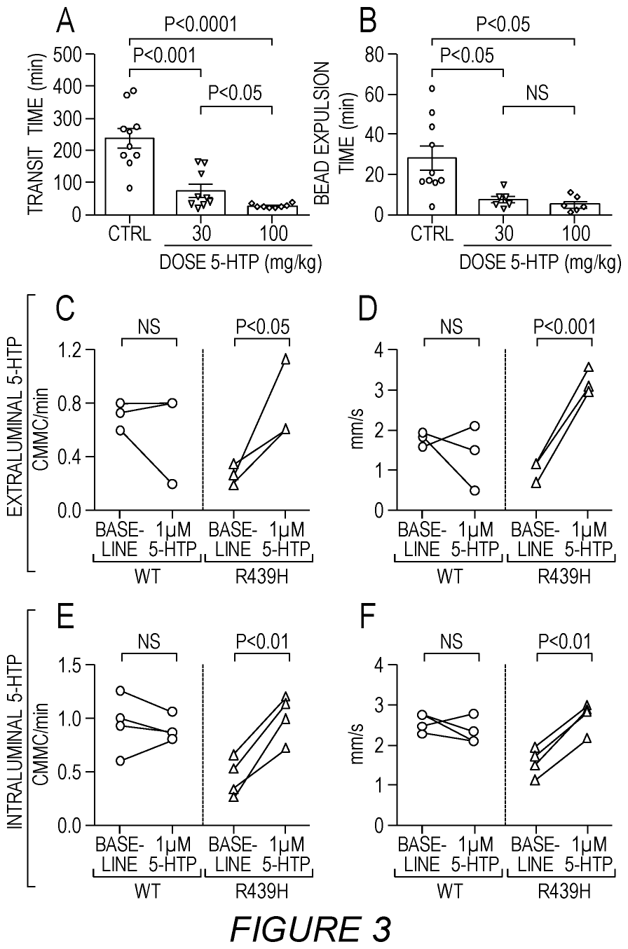

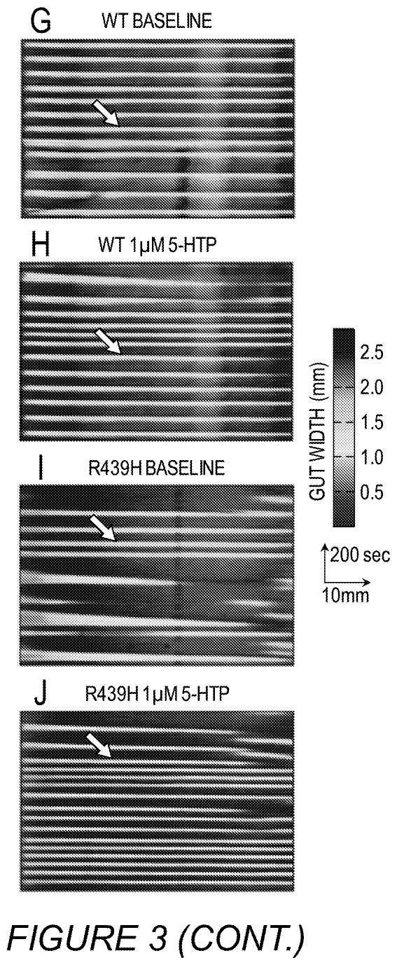

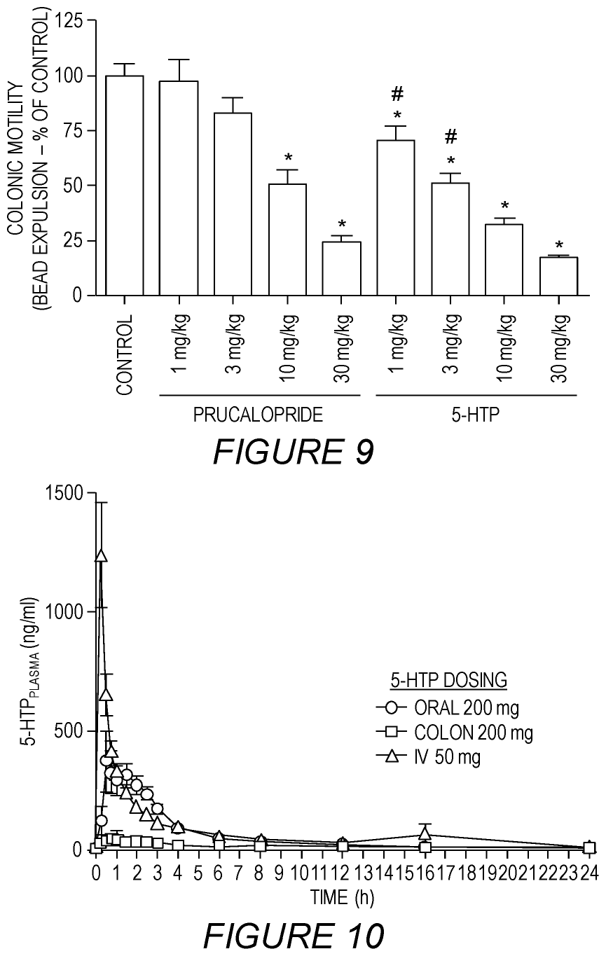

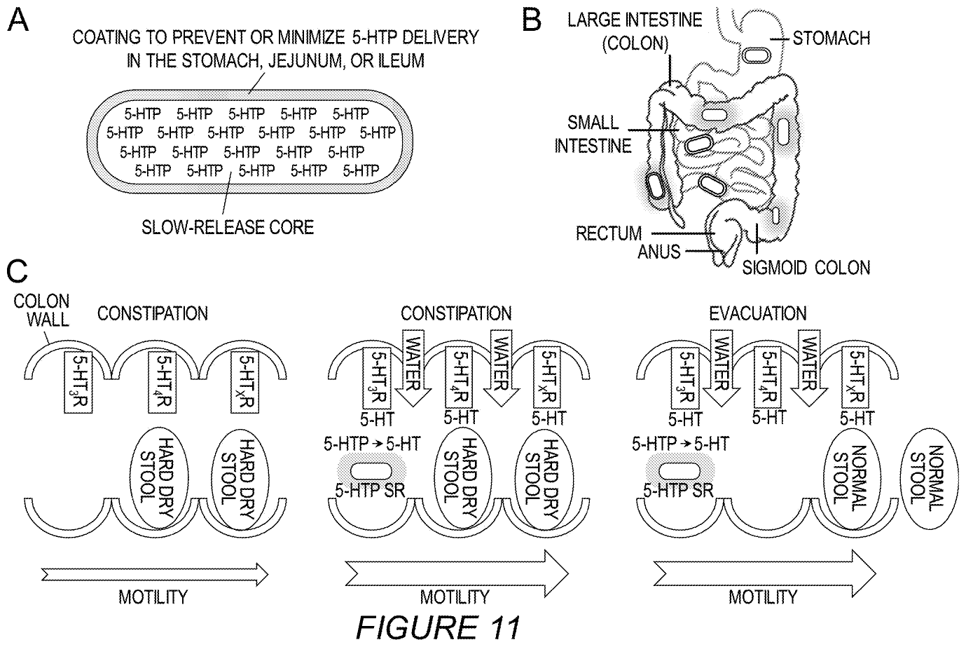

The data presented herein include the following: (i) In animals, 5-HTP enhances colonic motility, in vivo (FIGS. 3B, 8, and 9) and ex vivo (FIG. 3C-J). (ii) In animals, 5-HTP enhances colonic motility with higher efficacy (i.e. higher maximal effect) and higher potency (i.e. active at lower doses) compared to the specific 5-HT₄ receptor agonist prucalopride (marketed in the US as an anti-constipation drug as Motegrity®) (FIG. 8). (iii) 5-HTP administered as slow-release not only reverses constipation in a naturalistic animal model of constipation, but also reverses GI pathological changes implicated as casual in constipation (FIGS. 1, 3, 4, 5, 6, and 7). Thus, 5-HTP SR may treat both cause and symptoms in constipation. (iv) 5-HTP can reverse opioid-induced constipation (FIG. 8). (v) In humans, 5-HTP is minimally absorbed across the colonic intestinal wall (FIG. 10). This finding is unexpected, as previous rodent data found 5-HTP to substantially absorbed from the colon.

### Example 1: Mouse Proof-of-Concept Study of 5-HTP in Constipation Models

Introduction

Depression and constipation are common, potentially debilitating conditions that affect, respectively, 8% and 27% of the national population (Brody et al, 2018; Sanchez and Bercik, 2011). Although the two disorders can occur independently, depression is often comorbid with constipation. The prevalence of major depression in people with chronic constipation has been reported to be as high as 33% (Dipnall et al, 2016; Hosseinzadeh et al, 2011). Further, constipation is the leading comorbidity in depressed individuals (Hosseinzadeh et al, 2011).

Diseases that affect the brain, including autism spectrum disorder, Parkinson, and Alzheimer's disease are often associated with gastrointestinal (GI) disturbances (Rao and Gershon, 2016). Common pathophysiological mechanisms may account for the frequency of GI and CNS comorbidity in these conditions. This is not surprising because the structure of the enteric nervous system (ENS) resembles that of the CNS and the two systems utilize many of the same neurotransmitter mechanisms (Furness, 2008; Gershon, 2013b); moreover, the ENS and the CNS communicate with one another and the responsible nerves can be bidirectional conduits for disease transmission. The ENS, which is the largest and most complex unit of the peripheral nervous system, contains microcircuits that make it uniquely able to orchestrate GI behavior in the absence of CNS input; nevertheless, the normally occurring CNS-ENS intercommunication enables the two nervous systems to affect each other's function. A critical molecule that may participate, not only in ENS-CNS communication, but also in allowing the enteric microbiome to affect both, is serotonin (5-hydroxytryptamine; 5-HT).

5-HT acts as a neurotransmitter in both the CNS and the ENS (Gershon, 2013b); however, within the bowel, 5-HT is also found in enterochromaffin (EC) cells of the mucosal epithelium (Erspamer, 1953, 1966). Two different gene products, tryptophan hydroxylase 1 (TPH1) and tryptophan hydroxylase 2 (TPH2), catalyze the conversion of tryptophan to 5-hydroxytryptophan (5-HTP), which is rapidly converted by aromatic amino acid decarboxylase to 5-HT (Matthes and Bader, 2018). Two different 5-HT depots exist In the intestine: a large TPH1-dependent 5-HT pool in mucosal enterochromaffin (EC) cells and a much smaller TPH2-dependent pool in the serotonergic neurons of the ENS (Gershon and Tack, 2007).

Enteric 5-HT is a multifunctional molecule, with roles in paracrine, endocrine, and neurocrine signaling (Gershon, 2013b). Release of 5-HT from EC cells stimulates peristaltic and secretory reflexes and also is important in the transmission of sensory information, such as nausea and discomfort, to the CNS (Blackshaw and Grundy, 1993; Gregory and Ettinger, 1998; Grundy et al, 1994; Hagbom et al, 2011; Hillsley et al, 1998). In contrast, during development, and even in adult life, ENS-derived 5-HT stimulates ENS neurogenesis and has distinct effects that favor proliferation of late-developing neuronal subsets (e.g., dopamine- and GABA-expressing neurons) (Li et al, 2011b; Liu et al, 2009; Margolis et al, 2016c). Neuronal 5-HT also promotes intestinal mucosal growth and stimulates crypt epithelial cell proliferation (Gross et al, 2012; Margolis et al, 2016b). Although enteric mucosal and neuronal 5-HT both contribute to the regulation of GI motility, neuronal 5-HT plays a more prominent role in constitutive GI transit (Heredia et al, 2013; Li et al, 2011a; Margolis and Gershon, 2016a; Smith and Gershon, 2015). The functions of 5-HT, both in the local activity of the bowel and in signaling to the brain, highlight its potential involvement in the bidirectional communication between brain and gut that can simultaneously affect mood and GI motility (Cowen and Browning, 2015; Gaspar et al, 2003; Kendig et al, 2015). Notably, TPH2 is specifically required for CNS-derived 5-HT production (Matthes et al, 2018; Walther et al, 2003). Because TPH2 is essential for neuronal 5-HT biosynthesis in both the ENS and the CNS, it is likely to be involved in processes that concomitantly regulate CNS and ENS development and function.

Selective serotonin reuptake-inhibitors (SSRIs) are prescribed as first line-treatment for individuals with depression. SSRIs elevate brain extracellular levels of 5-HT by blocking reuptake through the serotonin reuptake transporter (SERT), leading to an increase in available 5-HT in the CNS (Stahl, 1998). Excess 5-HT in the CNS, in turn, promotes neurogenesis in depression-relevant regions such as the hippocampus and this has been hypothesized as a potential mechanism of action for its anti-depressant effects (Willner et al, 2013). SSRI treatment, however, induces remission in as few as a third of depressed patients (Jacobsen et al, 2016b; Trivedi et al, 2006). Further, the anti-cholinergic side effects of chronic SSRI therapy may worsen constipation (Marken and Munro, 2000). Thus, patients with depression are often faced with limited treatment options and prominent GI dysfunction.

Multiple coding variants of TPH2 are overexpressed in individuals with depression (Fasching et al, 2012; Karanovic et al, 2017; Tsai et al, 2009; Van der Auwera et al, 2014; Wigner et al, 2018; Zhang et al, 2005; Zill et al, 2004). One such mutation, a single nucleotide polymorphism in TPH2 in which a highly conserved Arg441 is replaced with His (G1463A; R441H), was shown to be 10-fold more prevalent in patients with SSRI-resistant unipolar depression than in individuals without depression (Zhang et al, 2005). Some individuals harboring the R441H mutation also exhibit symptoms consistent with anxiety (Zhang et al, 2005). Interestingly, the individuals expressing this mutation also exhibited a worsened depressive state upon treatment with an SSRI (Zhang et al, 2005). In order to determine whether SERT antagonism was inhibiting 5-HT production in these individuals, an analogous mutation to R441H, R439H, was knocked into a transgenic murine model and examined. The R439H mice exhibited a 60-80% decrease in CNS 5-HT levels as well as anxiety and depressive-like behaviors (Jacobsen et al, 2012a; Jacobsen et al, 2012b; Sachs et al, 2013a; Sachs et al, 2013b; Siesser et al, 2013; Zhang et al, 2005). The phenotype of the R439H mice thus suggests that the constitutively decreased activity of TPH2 in these animals interferes with the 5-HT signaling required for normal CNS development and function. Further, treatment of the R439H mice with the SSRI, fluoxetine worsened their depressive behaviors and resulted in a further decline in CNS 5-HT levels. Because TPH2, 5-HT and SERT are also present in the intestine, it is possible that the R439H mutation may not only cause abnormalities in the CNS but also in ENS development and function.

The relationship between the brain and the intestine may also be modulated by the enteric microbiota (Burokas et al, 2015; Dinan and Cryan, 2017; Kelly et al, 2016b; Yano et al, 2015), the >100 trillion microbes within the intestine that influence ENS and CNS development (O'Mahony et al, 2015; Obata and Pachnis, 2016; Sampson and Mazmanian, 2015), GI motility (Quigley and Spiller, 2016; Reigstad et al, 2015; Tigchelaar et al, 2016) and mood (Kelly et al, 2016a; Yarandi et al, 2016). Associations between specific intestinal bacteria with depression and/or constipation have been described (Jiang et al, 2015; Lin et al, 2017) (Strati et al, 2017; Wang et al, 2017; Zhu et al, 2014; Zoppi et al, 1998) (Huang et al, 2018). Further, a bidirectional relationship may exist between the specific enteric microbiota, their metabolites and 5-HT (Wikoff et al, 2009) (Reigstad et al, 2015) (Yano et al, 2015) (Oleskin et al, 1998; Tsavkelova et al, 2006). Specific enteric microbiota might, therefore, also play a role in the link between constipation and depression.

If a genetic variant that causes TPH2 hypoefficiency results in 5-HT dysfunction and, ultimately, in constipation and depression, a reasonable approach for treatment might be to increase ENS and non-ENS 5-HT in the gut while bypassing TPH2. We thus sought to identify a novel therapeutic approach that could successfully treat both depression and GI abnormalities effectively and simultaneously. We considered 5-hydroxytryptophan (5-HTP), a therapeutically relevant precursor of 5-HT (Jacobsen et al, 2016b). Acute adjunct 5-HTP has been reported to enhance the effects of SSRI treatment in human and animal models, to improve the efficacy of SSRI therapy in SSRI-resistant depression and to prevent the exacerbation of the 5-HT deficiency that results in TPH2 R441H carriers on SSRI therapy (Jacobsen et al, 2016a; Jacobsen et al, 2016b; Nardini et al, 1983; Siesser et al, 2013). The rapid absorption and elimination profile of acute adjunct 5-HTP, however, reduces its ability to maintain the sustained levels of 5-HT necessary for effectual treatment.

Recent advances in pharmacotherapeutic design have enabled the production of a sustained release formulation of 5-HTP (5-HTP SR). In contrast to immediate release 5-HTP, 5-HTP SR maintains therapeutically relevant levels of CNS 5-HT in animal models of S SRI-resistant depression (Jacobsen et al, 2016a; Jacobsen et al, 2016b).

We tested hypotheses that: (1) Changes in TPH2-mediated 5-HT production that accompany depression also contribute to abnormalities in: (a) enteric neuronal development, (b) ENS-mediated GI functions and (c) anomalies in the enteric microbiota that accompany those demonstrated in individuals with depression and/or constipation and (2) That these abnormalities can be treated through mechanism-guided intervention with 5-HTP SR.

We found that the R439H animals, that harbor S SRI-resistant anxiety- and depressive-like behaviors, also manifest significant abnormalities in enteric neuronal development, regulation of ENS-modulated functions including GI motility and enteric mucosal growth as well as abnormalities in the enteric microbiome that mimic those seen in individuals with depression and/or constipation. Strikingly, we also found that these abnormalities could be effectively treated with 5-HTP SR. Our findings reveal that not only CNS manifestations, but also adult intestinal neuroanatomy and long-term ENS-guided function are exquisitely sensitive to TPH2-mediated 5-HT activity during ontogeny and, further, that these abnormalities can be reversed with a novel sustained-release version of 5-HTP during adulthood.

Results:

Numbers of Enteric Neurons are Related to TPH2 Activity During Development.

TPH2 synthesizes 5-HT in the ENS and 5-HT promotes enteric neuronal development; therefore, we tested the hypothesis that the TPH2 R439H mutation, which causes a decrease in 5-HT production, would impede enteric neurogenesis (Li et al, 2011a; Liu et al, 2009). Total numbers of enteric neurons and later-developing neuronal subsets, shown previously to be under serotonergic control, were identified immunocytochemically and quantified in whole mounts of laminar preparations dissected from the gut wall of mice at 10-12 weeks of age. Because serotonergic neurons are born early and 5-HT enhances development of late-born neurons expressing tyrosine hydroxylase (TH) (dopaminergic neurons) and γ-aminobutyric acid (GABA) (Li et al, 2011a; Margolis et al, 2016b), these phenotypes were chosen for study. Neuronal numbers were quantified as a function of ganglionic area, which is a parameter that is relatively resistant to stretching of the tissue (Karaosmanoglu et al, 1996). R439H mice were compared with WT littermates. GABA-expressing neurons were quantified in the myenteric plexus where they are located, whereas TH-expressing neurons were quantified in the submucosal plexus where most are found.

Total enteric neurons were significantly less abundant in R439H mice than in WT littermates (FIG. 1). This difference was observed in both the myenteric plexus (FIG. 1A: 68.7%±10.3% of WT, P<0.05, n=4; also compare I with L) and submucosal plexus (FIG. 1C: 66.3%±5.0% of WT, P<0.05, n=3) of the ileum, and in the myenteric plexus (FIG. 1E: 61.6%±7.5% of WT, P<0.05, n=4) and submucosal plexus (FIG. 1G: 60.5%±6.1% of WT, P<0.05, n=4; also compare O with R) of the colon. Late-born neuronal phenotypes were more sensitive to the level of TPH2 activity during development than total neurons and thus appear to be selectively affected. In R439H mice, myenteric GABA-expressing neurons were deficient not only in absolute numbers (FIG. 1A: ileum, 17.2%±6.3% of WT, P<0.0005, n=4; FIG. 1E: colon, 49.9%±4.8% of WT, P<0.0005, n=4) but also as a proportion of total neurons (FIG. 1B: ileum, 8.7%±2.5% in WT vs 2.2%±0.4% in R439H, P<0.05, n=4; FIG. 1F: colon, 7.1%±0.1% in WT vs 5.7%±0.5% in R439H, P<0.05, n=4; also compare J with M) in both the ileum and colon. Submucosal TH-expressing neurons were also deficient in absolute numbers in the ileum (FIG. 1C: 72.6%±2.6% of WT, P<0.05, n=4) and colon (FIG. 1G: 35.5%±8.2% of WT, P<0.05, n=4), however, the percentage of TH-expressing neurons as a proportion of total neurons was unaffected in the ileum (FIG. 1D: 27.3%±1.0% in WT vs 27.4%±2.4% in R439H, P=0.97, n=4; also see P and S) and the colon (FIG. 1H: 10.6%±2.6% in WT vs 5.9%±1.5% in R439H, P=0.17, n=4). Expression of the R439H mutation thus leads to a hypoplastic ENS with selective impairment of development/survival of late-born neurons, the development of which are 5-HT-promoted.

The TPH2 R439H Mutation Leads to Slow GI Transit and Impairment of the Peristaltic Reflexes.

Experiments were implemented to determine whether the long-lasting ENS hypoplasia associated with the R439H mutation is reflected in GI motility. In mice 10-12 weeks of age, measurements were made in vivo of total GI transit time, propulsive colorectal motility, gastric emptying, and upper intestinal transit. Further, colonic migrating motor complexes (CMMCs; peristaltic reflexes) were investigated in vitro to determine whether changes in intestinal transit in R439H mice are due to an intrinsic defect of the ENS. The extrinsic innervation of the gut is severed in isolated preparations, and the intrinsic circuits of the ENS mediate CMMCs (Spencer et al, 1998).

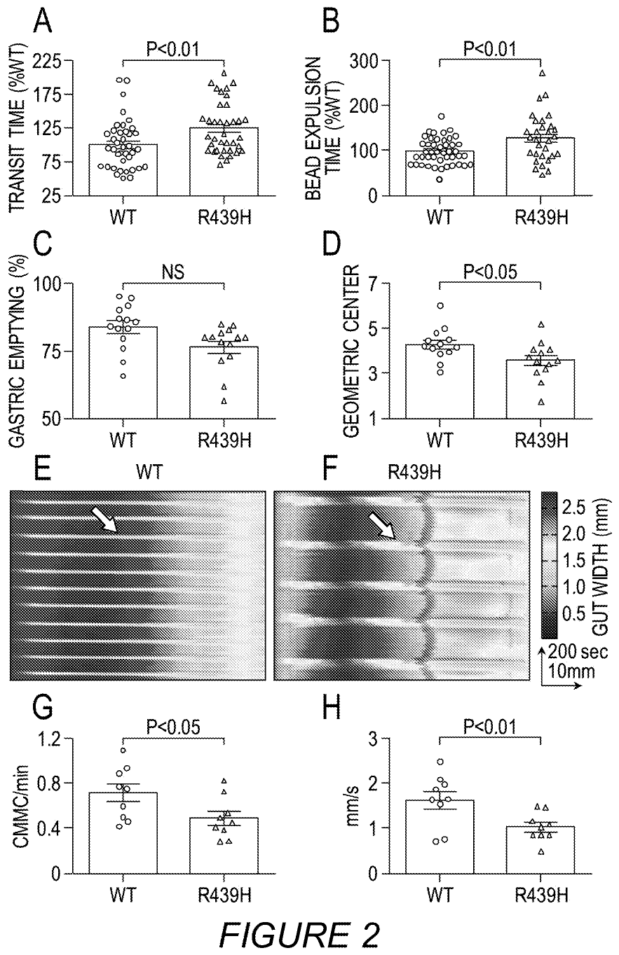

Total GI transit time (FIG. 2A: 124.1%±6.2% of WT, P<0.01, n=37-39), propulsive colorectal motility (FIG. 2B: 125.9%±9.1% of WT, P<0.01, n=32-43), and upper intestinal transit (FIG. 2D: as geometric center, 4.3±0.2 in WT vs 3.6±0.2 in R439H, P<0.05, n=13) were all significantly slower in R439H mice than in WT littermates. In contrast, the rate of gastric emptying (FIG. 2C: as % gastric emptying, 83.8%±2.3% in WT vs 76.8%±2.2% in R439H, P=0.06, n=13) in R439H and WT animals did not differ significantly.

To measure in vitro motility, intraluminal pressure was raised in isolated preparations of colon to initiate CMMCs, preparations were video-imaged, and spatiotemporal maps of contractile activity patterns were constructed (FIG. 2E, 2F). Analysis of these maps revealed that CMMC frequency (FIG. 2G: 0.71±0.08 CMMC/min in WT vs 0.49±0.06 CMMC/min in R439H, P<0.05, n=9) and velocity (FIG. 2H: 1.6±0.2 mm/s in WT vs 1.0±0.1 mm/s in R439H, P<0.01, n=9) were significantly lower in R439H than in WT mice, while conduction length was not altered (data not shown). These observations suggest that the ENS hypoplasia of R439H mice impairs generation and conduction of peristaltic reflexes.

Immediate-Release (IR) 5-HTP Increases In Vivo Motility and In Vitro Peristaltic Contractions.

The R439H mutation results in less neuronal 5-HT production. Increasing the 5-HT available for neurotransmission may thus ameliorate the defects in ENS development and GI function. 5-HTP is a therapeutically relevant precursor of 5-HT and has previously been shown to increase intestinal motility (Bogdanski et al, 1958; Bueno and Fioramonti, 1982; Gorard et al, 1994; Schemann and Ehrlein, 1986; Wang et al, 2007a). In order to determine whether 5-HTP can increase in vivo motility, and whether this occurs by ENS stimulation, we examined whether immediate-release 5-HTP (5-HTP IR) could increase in vivo TGIT and colonic motility as well as the frequency and/or speed of CMMCs, respectively. To conduct these studies, 30 and 100 mg/kg doses of 5-HTP IR were administered intraperitoneally to WT mice. 5-HTP IR significantly decreased total in vivo GI transit time in a dose-dependent manner (FIG. 3A: 238.7±29.6 min in Control vs 74.9±19.5 min in 30 mg/kg vs 25.4±2 7 min in 100 mg/kg, ANOVA P<0.0001, n=8-10). Further, either dose of 5-HTP IR significantly decreased the time of colonic bead expulsion (FIG. 3B: 28.5±5.9 min in Control vs 7.2±1.6 min in 30 mg/kg vs 5.2±1.6 min in 100 mg/kg, ANOVA P<0.01, n=6-10).

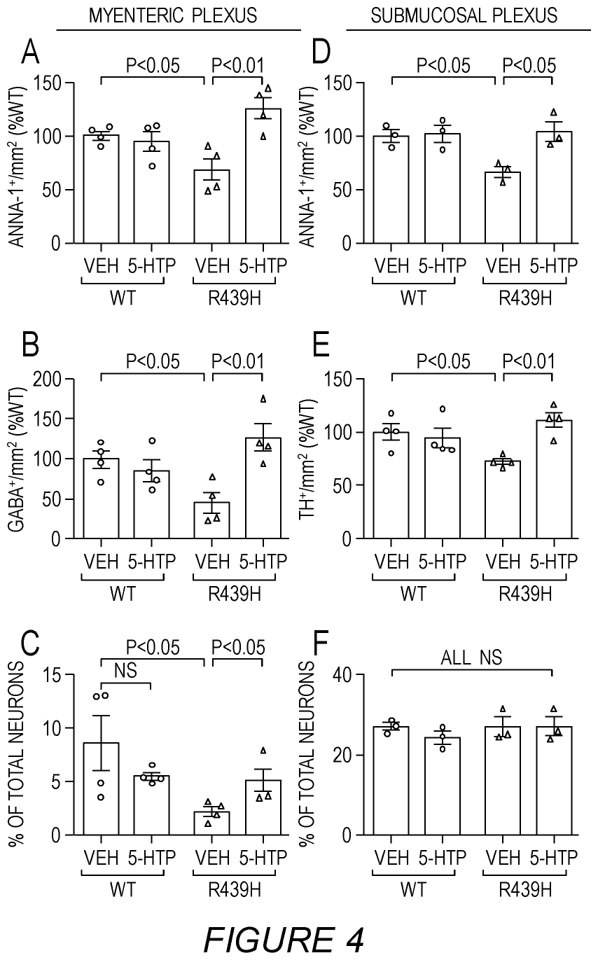

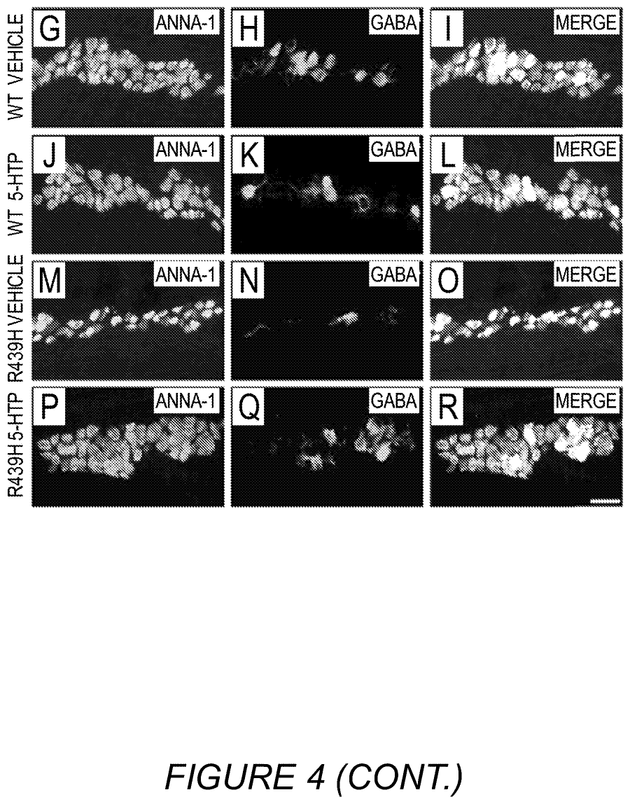

To determine the effect of 5-HTP IR on in vitro peristaltic contractions (CMMCs), which reflect the intrinsic behavior of the ENS, isolated preparations of colon were used to generate spatiotemporal maps of contractile activity patterns before and after administration of 5-HTP IR both intra- and extra-luminally (FIG. 3G-J). Dose response curves were initially conducted to determine a dose of 5-HTP whereby GI motility would both decrease in R439H mice and not significantly alter motility in WT mice. We found that, in R439H mouse colons exposed to an extraluminal 1 μM dose of 5-HTP IR, CMMC frequency (FIG. 3C: 0.27±0.04 CMMC/min in baseline vs 0.78±0.18 CMMC/min in 5-HTP, P<0.05, n=3) and CMMC velocity (FIG. 3D: 1.0±0.2 mm/s in baseline vs 3.2±0.2 in 5-HTP, P<0.001, n=3) were both significantly increased, while both parameters remained unchanged in WT mice (FIG. 3C: 0.71±0.06 CMMC/min in baseline vs 0.60±0.20 CMMC/min in 5-HTP, P=0.62, n=3; FIG. 3D: 1.8±0.1 mm/s in baseline vs 1.4±0.5 mm/s in 5-HTP, P=0.42, n=3). Similarly, in R439H mouse colons that were exposed to an intraluminal 1 μM dose of 5-HTP IR, there was a subsequent increase in CMMC frequency (FIG. 3E: 0.45±0.09 CMMC/min in baseline vs 1.02±0.10 in 5-HTP, P<0.01, n=4) and CMMC velocity (FIG. 3F: 1.5±0.2 mm/s in baseline vs 2.7±0.2 mm/s in 5-HTP, P<0.01, n=4). Both parameters were again unchanged in WT mice (FIG. 3E: 0.95±0.14 CMMC/min in baseline vs 0.90±0.06 CMMC/min in 5-HTP, P=0.75, n=4; FIG. 4F: 2.6±0.1 minis in baseline vs 2.3±0.2 mm/s in 5-HTP, P=0.23, n=4). These observations suggest that 5-HTP accelerates total GI transit and colonic motility by increasing peristaltic contraction velocity and frequency, therefore acting through mechanisms that reflect increased ENS activation.

Administration of Sustained-Release 5-HTP (5-HTP SR) During Adulthood Rescues Mice from the ENS Abnormalities Associated with the R439H Mutation.

5-HT increases enteric neurogenesis, at least in part by stimulation of 5-HT₄ receptors (Liu et al, 2009). If TPH2 hypoactivity were to impede enteric neurogenesis by diminishing 5-HT availability, an exogenously supplied source of 5-HT that is not a substrate for TPH2, nor inactivated quickly by SERT, ought to countermand this defect. 5-hydroxytryptophan (5-HTP) is a 5-HT precursor that can elevate levels of 5-HT (Jacobsen et al, 2016a). The rapid absorption and elimination of 5-HTP by SERT, however, interferes with the ability to maintain therapeutically relevant levels of adjunct 5-HTP. 5-HTP SR significantly enhances 5-HTP concentration in the CNS, as well as the treatment efficacy of depression, in mouse models. We therefore tested the idea that oral administration of 5-HTP SR would also rectify the abnormalities in GI neuroanatomy and, consequently, improve ENS-mediated defects in the R439H mice. 5-HTP SR, in powdered form, was incorporated into mouse chow. Mice received approximately 1 g/kg/day (6.7 mg 5-HTP per gram food), as previously described (Jacobsen et al, 2016b) beginning at 6-7 weeks of age for 4 weeks.

In the R439H mice, four weeks of 5-HTP SR ameliorated the hypoplasia that occurred in total neurons (ANNA-1-immunoreactive) in both the myenteric plexus (FIG. 4A: as % of WT, 68.7%±10.3% in R439H vs 125.7%±9.9% in R439H with 5-HTP, P<0.01, n=4, 1-way ANOVA P<0.005) and the submucosal plexus (FIG. 4D: as % of WT, 66.3%±5.0% in R439H vs 104.3%±9.3% in R439H with 5-HTP, P<0.05, n=3, 1-way ANOVA P<0.05) of the ileum. 5-HTP SR treatment also reversed the R439H-associated deficiencies in absolute numbers of myenteric GABA-expressing neurons (FIG. 4B: as % of WT, 46.5% 12.9% in R439H vs 127.6%±17.5% in R439H with 5-HTP, P<0.01, n=4, 1-way ANOVA P<0.01) and dopaminergic neurons (TH-immunoreactive; FIG. 4E: as % of WT, 72.6%±2.6% in R439H vs 111.6%±7.0% in R439H with 5-HTP, P<0.005, n=4, 1-way ANOVA P<0.05). Further, 5-HTP SR treatment in R439H mice increased the proportion of GABA-expressing neurons in the myenteric plexus (FIG. 4C: 2.2%±0.4% in R439H vs 5.2%±1.0% in R439H with 5-HTP, P<0.05, n=4, 1-way ANOVA P<0.05), while the proportion of dopaminergic neurons in the myenteric plexus was unaffected (FIG. 4F: 27.4%±2.4% in R439H vs 27.3%±2.3% in R439H with 5-HTP, P=0.99, n=3, 1-way ANOVA P=0.65). 5-HTP SR treatment in WT littermates did not affect numbers of total neurons in the myenteric (FIG. 4A: as % of WT, 101.1%±3.9% in WT vs 94.6%±8.8% in WT with 5-HTP, P=0.53, n=4) or submucosal plexuses (FIG. 4D: as % of WT, 100.0%±5.9 in WT vs 102.2%±8.1% in WT with 5-HTP, P=0.84, n=3), GABAergic neurons in the myenteric plexus (FIG. 4B: as % of WT, 100.0%±10.8% in WT vs 86.7%±13.3% in WT with 5-HTP, P=0.47, n=4), dopaminergic neurons in the submucosal plexus (FIG. 4E: as % of WT, 100.0%±7.8% in WT vs 94.7%±9.2% in WT with 5-HTP, P=0.68, n=4), nor the proportions of either GABAergic neurons (FIG. 4C: 8.7%±2.6% in WT vs 5.6%±0.4% in WT with 5-HTP, P=0.27, n=4) or dopaminergic neurons as a ratio of total neurons (FIG. 4F: 27.3%±1.0% in WT vs 24.5%±1.6% in WT with 5-HTP, P=0.20, n=3).

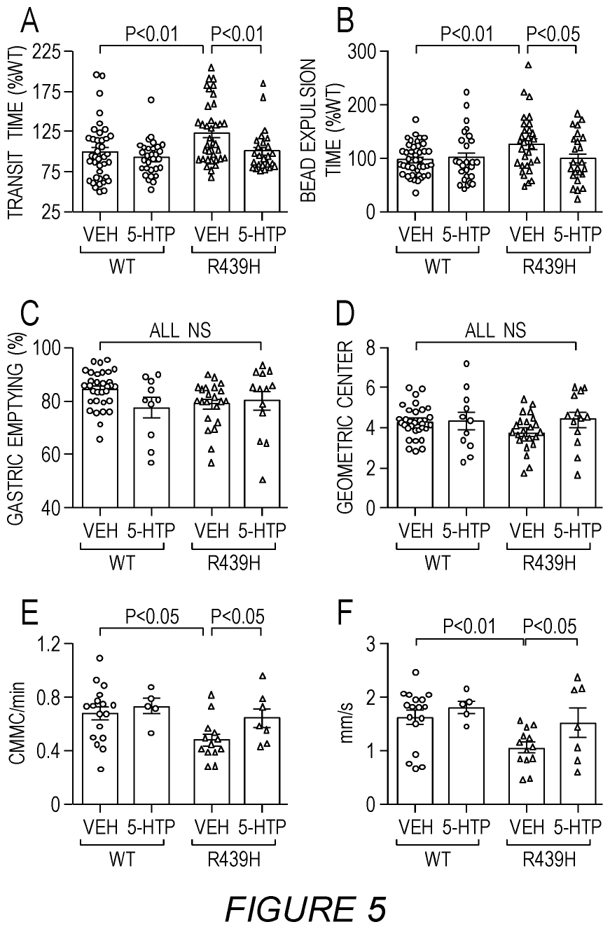

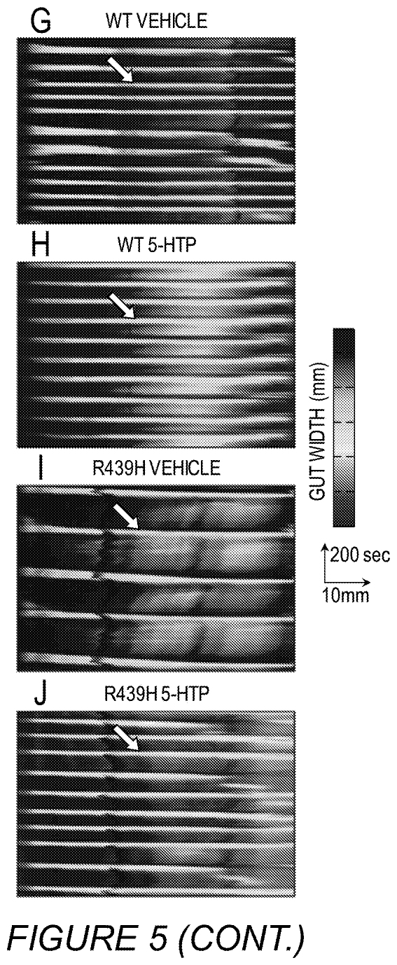

The normalization of neuronal numbers in the myenteric and submucosal plexuses that accompanied 5-HTP SR administration was partnered with rescue from the R439H-associated slowing of total GI transit time (FIG. 5A: as % of WT, 124.1%±6.2% in R439H vs 101.5%±5.1% in R439H with 5-HTP, P<0.01, n=27-37, 1-way ANOVA P<0.001) and colonic motility (FIG. 5B: as % of WT, 125.9%±9.1% in R439H vs 98.9%±9.0% in R439H with 5-HTP, P<0.05, n=25-32, 1-way ANOVA P<0.05). In contrast, 5-HTP SR did not affect gastric emptying (FIG. 5C: as % gastric emptying, 78.8%±1.7% in R439H vs 79.9%±3.6% in R439H with 5-HTP, P=0.57, n=13, 1-way ANOVA P=0.08) or upper intestinal transit (FIG. 5D: as geometric center, 3.8±0.2 in R439H vs 4.4±0.4 in R439H with 5-HTP, P<0.05, n=13, 1-way ANOVA P=0.21). 5-HTP SR treatment in WT littermates did not significantly affect total GI transit (FIG. 5A: as % of WT, 100.0%±5.8% in WT vs 93.1%±4.0% in WT with 5-HTP, P=0.36, n=30-39), colonic motility (FIG. 5B: as % of WT, 96.2%±4.6% in WT vs 101.6%±9.1% in WT with 5-HTP, P=0.56, n=27-43), gastric emptying (FIG. 5C: as % gastric emptying, 84.3%±1.3% in WT vs 77.4%±3.7% in WT with 5-HTP, P=0.06, n=10), or upper intestinal transit (FIG. 5D: as geometric center, 4.3±0.1 in WT vs 4.3±0.5 in WT with 5-HTP, P=0.57, n=11).

The 5-HTP SR-mediated rescue of in vivo motility was also manifest in a normalization of CMMCs measured in vitro; 5-HTP SR treatment normalized the R439H-associated decreases in CMMC frequency (FIG. 5E: 0.48±0.04 CMMC/min in R439H vs 0.64±0.07 CMMC/min in R439H with 5-HTP, P<0.05, n=7, 1-way ANOVA P<0.05) and velocity of propagation (FIG. 5F: 1.0±0.1 mm/s in R439H vs 1.5±0 3 mm/s in R439H with 5-HTP, P<0.05, n=7, 1-way ANOVA P<0.01). Spatiotemporal maps are demonstrated in FIG. 5G-J. Treatment of WT littermates with 5-HTP SR did not significantly alter either CMMC frequency (FIG. 5E: 0.68±0.05 CMMC/min in WT vs 0.73±0.06 CMMC/min in WT with 5-HTP, P=0.78, n=5) or CMMC velocity (FIG. 5F: 1.6±0.1 minis in WT vs 1.8±0.1 mm/s in WT with 5-HTP, P=0.33, n=5). These in vitro observations suggest that the normalization of GI motility induced by 5-HTP SR is due to a normalization of ENS function.

The R439H Mutation Leads to Decreased Villus Height and Crypt Perimeter that is Ameliorated by Administration of 5-HTP SR.

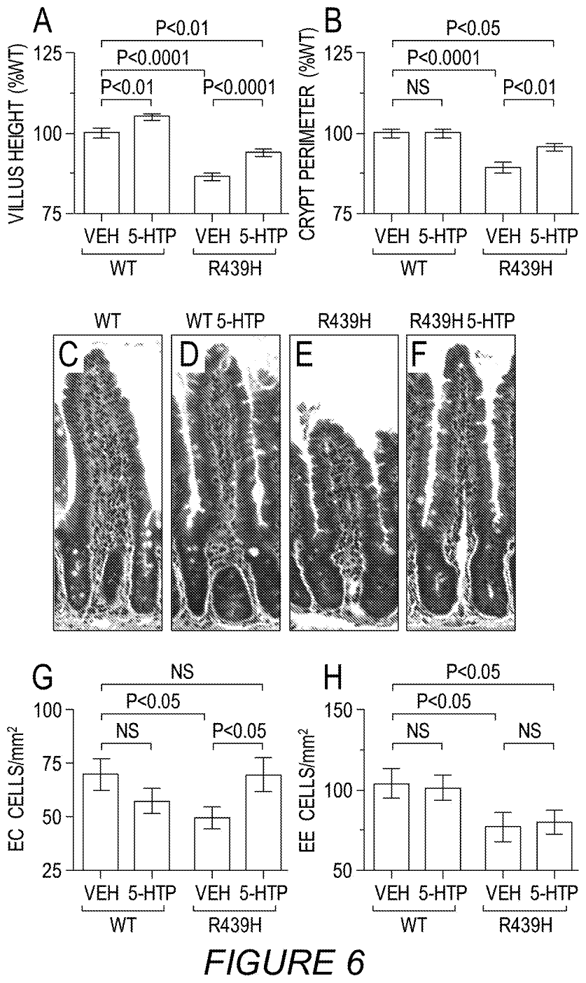

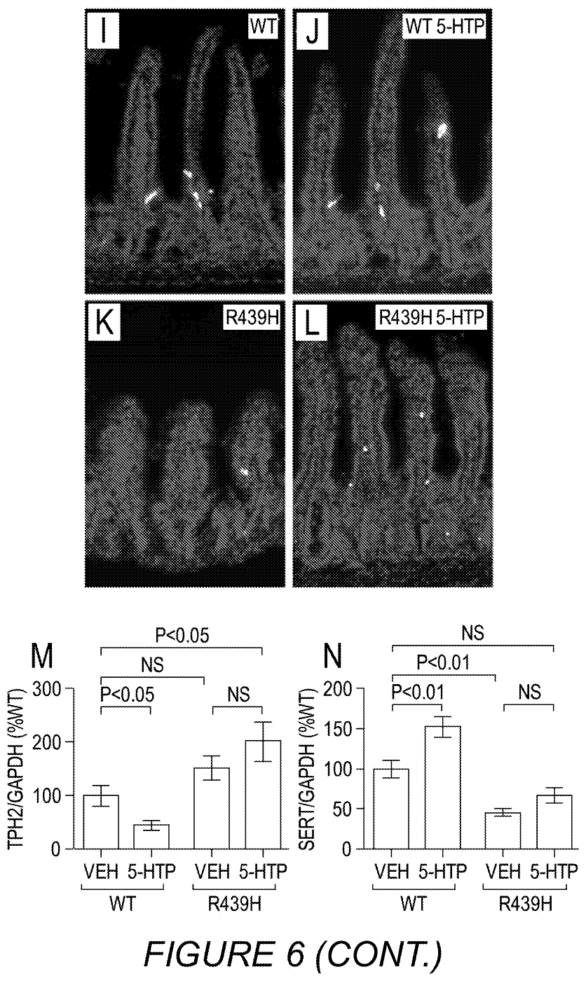

TPH2 regulation of 5-HT signaling is important for epithelial balance. The ENS, and specifically 5-HT, has been linked to mucosal maintenance (Gross et al, 2012); myenteric serotonergic neurons innervate submucosal cholinergic neurons that regulate proliferation of transit-amplifying cells. As a result, the cell proliferation index, crypt depth, and villus height are all greater in SERTKO than in WT mice and are deficient in TPH2KO mice (Gross et al, 2012). Neuronal, rather than mucosal 5-HT has been shown to mediate these effects (Gross et al, 2012). We therefore compared villus height and crypt perimeter in R439H mice constitutively as well as after chronic treatment with 5-HTP SR (FIG. 6). Villus height and crypt perimeter in the upper intestine were significantly decreased in R439H mice relative to WT (FIG. 6A: 86.2%±1.2% of WT, P<0.0001, n=4-5, 1-way ANOVA P<0.0001; FIG. 6B: 89.2%±1.6% of WT, P<0.0001, n=4-5, 1-way ANOVA P<0.0001). Treatment with 5-HTP SR increased villus height in both R439H and WT mice (FIG. 6A: 105.5%±0.9% of WT in WT with 5-HTP, P<0.01, n=4-5; 93.7%±1.0% of WT in R439H with 5-HTP, P<0.0001 vs R439H, n=4-5). Further, in R439H mice, treatment with 5-HTP increased the perimeter of upper intestinal crypts (FIG. 6B: 95.7%±1.2% of WT in R439H with 5-HTP, P<0.01 vs R439H, n=4-5), while there was no difference in crypt perimeter in WT mice treated with 5-HTP (FIG. 6B: 100.1%±1.3% of WT in WT with 5-HTP, P=0.97, n=4-5). These data are consistent with prior reports demonstrating that villus and crypt measurements are also significantly decreased in in TPH2KO mice (Gross et al, 2012). Representative villi and crypts are demonstrated in FIG. 6C-F.

There were also effects of the R439H mutation on cell lineage. Interestingly, the relative density of EC cells and that of the entire enteroendocrine (EE) cell class were significantly less in R439H than in WT mice (FIG. 6G: 69.8±7.4 cells/mm² in WT vs 49.4±5.1 cells/mm² in R439H, P<0.05, n=4-5, 1-way ANOVA P<0.05; FIG. 6H: 104.1±9.4 cells/mm² in WT vs 77.1±9.1 cells/mm² in R439H, P<0.05, n=4-5, 1-way ANOVA P<0.05). The relative density of EC cells significantly increased in the R439H mice after chronic administration of 5-HTP SR (FIG. 6G: 69.7±8.1 cells/mm² in R439H with 5-HTP vs 49.4±5.1 cells/mm² in R439H, P<0.05, n=4-5) while it was unaltered in WT mice that received the drug (FIG. 6G: 57.6±5.9 cells/mm² in WT with 5-HTP vs 69.8±7.4 cells/mm² in WT, P=0.20, n=4-5). Treatment with 5-HTP SR did not, however, have an effect on relative density of EE cells in either WT (FIG. 6H: 101.6±7.9 cells/mm² in WT with 5-HTP, P=0.84 vs WT, n=4-5) or R439H mice (79.9±7.6 cells/mm² in R439H with 5-HTP, P=0.82 vs R439H, n=4-5). Representative stains of enterochromaffin cells in the upper intestine, labeled with 5-HT, are shown in FIG. 6I-L. These findings are consistent with the idea that there may be a level of 5-HT at which cell lineage stops responding to increases in available 5-HT, as the changes seen in EC and EE cell number after 5-HTP SR administration were not found in the WT mice that received 5-HTP SR.

Transcription of Tph2 and SERT are Altered in R439H Mice Before or after Chronic Administration of 5-HTP SR.

Experiments were carried out to determine whether the hypoactive TPH2 of R439H mice altered transcription of Tph2, SERT and DAT. In the intestines of R439H mice, transcripts encoding TPH2 were not significantly different constitutively than in those of WT animals (FIG. 6M: 150%±22% of WT, P=0.09, n=18-21, 1-way ANOVA P<0.0005). In contrast, in the upper intestines of R439H mice and WT mice that received four weeks of 5-HTP SR, transcripts encoding TPH2 were significantly decreased in the WT mice that received 5-HTP SR (FIG. 6M: 44%±9% of WT, P<0.05, n=14-21) but were unchanged in R439H mice that received 5-HTP SR (FIG. 6M: 201%±38% of WT, P=0.24 vs R439H, n=15-18). Further, R439H mice that received 5-HTP SR had significantly greater levels of TPH2 transcripts than either the WT control mice or the WT mice that received 5-HTP SR (FIG. 6M: 201%±38% of WT, P<0.05 vs WT, P<0.0006 vs WT with 5-HTP, n=14-21).

In contrast to TPH2, SERT transcript numbers were significantly lower in the R439H mice constitutively compared to WT mice (FIG. 6N: 45%±5% of WT, P<0.0005, n=17-23, 1-way ANOVA P<0.0001). Interestingly, levels of SERT transcription did not change in the R439H mice after chronic administration of 5-HTP SR (FIG. 6N: 45%±5% of WT in R439H vs 67%±10% of WT in R439H with 5-HTP, P=0.07, n=17-18) while levels increased significantly in the WT mice that received the drug for a similar period of time (FIG. 6N: 152%±13% of WT in WT with 5-HTP, P<0.01 vs WT, n=19-23). These findings are consistent with the idea that SERT is regulated in response to available 5-HT levels, as it was shown to be decreased in response to the decreased levels of 5-HT in R439H mice and increased in WT mice that received 5-HTP SR. DAT was not found to be significantly different in any of the groups (not shown) indicating that it does not increase to compensate for the decreased levels of SERT transcription present in the R439H mice.

Abnormalities in the Enteric Microbiome of the R439H Mice Mimic Those Seen in Individuals with Constipation and/or Depression.

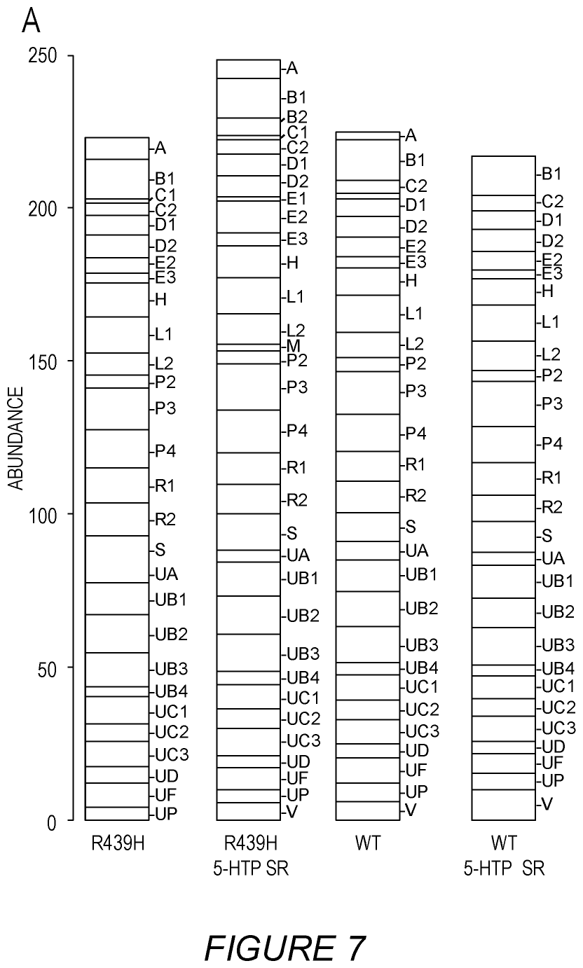

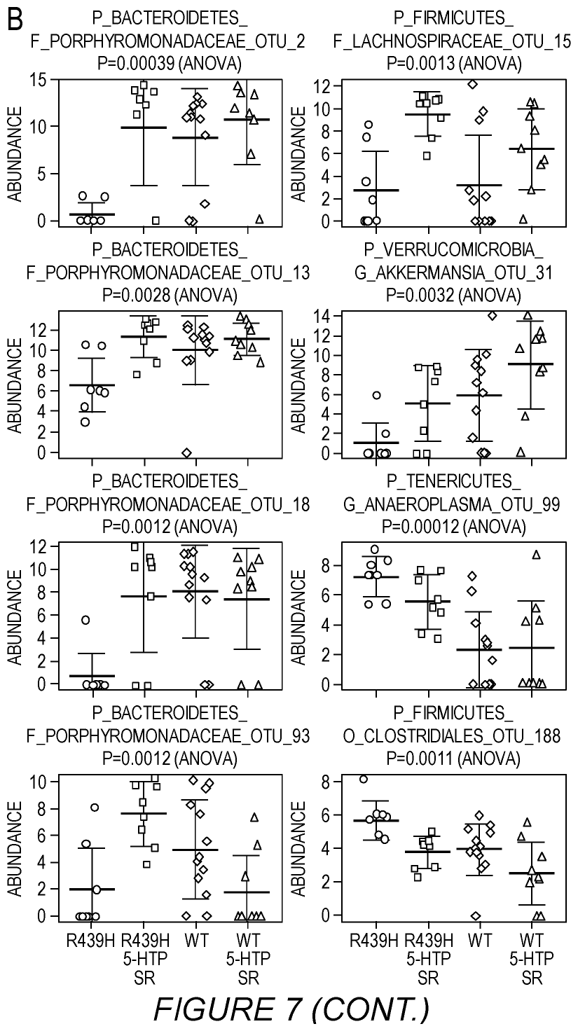

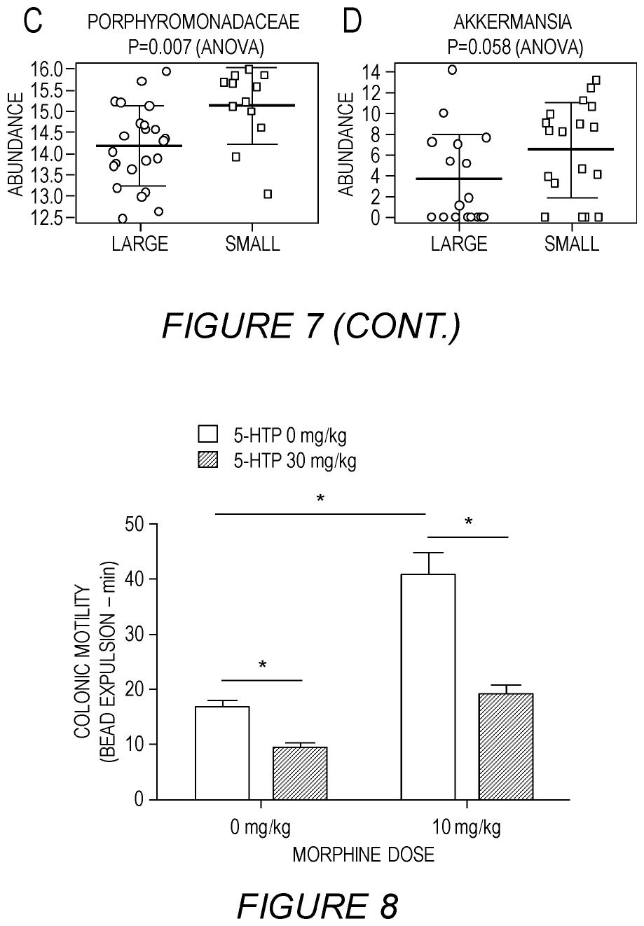

The GI microbiome has been associated with changes in mood and GI motility and may thus be an important link between depression and constipation. We therefore conducted deep sequencing of feces from the R439H and WT mice with and without 5-HTP SR treatment. After evaluation of the microbiome, integrative analyses were conducted to determine if any specific microbiota were associated with changes in motility or neuroanatomy parameters. No significance differences were observed regarding bacterial diversity amongst all groups (not shown). The enteric microbiome in all mice was mainly comprised of Bacteroidetes, Firmicutes, and Proteobacteria. Family-level analysis (FIG. 7A) showed decreases in several families in the R439H mice compared to WT that recovered following administration of 5HTP SR; Anaeroplasmataceae (p=0.00039) was increased in R439H mice and decreased to levels congruent with WT mice after receipt of 5-HTP SR (Supp. FIG. 1). Further, families that were originally decreased in R439H compared to WT but increased to WT levels following 5-HTP SR administration included: Lactobacillaceae (p=0.031), Porphyromonadaceae (p=0.04), Sutterellaceae (p=0.0045), and Verrucomicrobiaceae (p=0.0016) (FIG. 7B, Supp FIG. 1). Relevant OTUs contributing to these differences included several types of Porphyromonadaceae (OTU_2 [p=0.00039], OTU_13 [p=0.0028], OTU_18 [p=0.0012], and OTU_93 [p=0.0012]), Lachnospiraceae (OTU_15 [p=0.0013] and OTU_188 [p=0.0011]), Anaeroplasmataceae (OTU_99 (p=0.00012), and Verrucomicrobiaceae (OTU_31 (p=0.0032) (FIG. 7A,B, Supp FIG. 1). Of note, the most pronounced finding was a significantly decreased abundance of Verrucomicrobia (p=0.0017) in the R439H mice constitutively compared to all other groups. OTU_31, identified as Akkermansia muciniphila, singularly and heavily contributed to the differences observed in abundance of Verrucomicrobia, specifically Verrucomicrobiaceae (FIG. 7A,B).

Integrative analyses were conducted to evaluate associations between specific microbiota, ENS neuroanatomy and ENS function. Family-level analysis revealed increases in Porphyromonadaceae (p=0.007) and Sutterellaceae (p=0.038) in mice with slower colonic motility (FIG. 7C). Slower total GI transit time was associated with a trend toward increased Verrucomicrobiaceae, specifically Akkermansia sp. (p=0.058) (FIG. 7D). A normalcy in enteric neuron numbers was observed in concert with trends toward increases in several families, including Porphyromonadaceae (p=0.093), Sutterellaceae (p=0.11), and Verrucomicrobiaceae (p=0.10) but none of these reached significance (not shown).

Methods:

Animals.

TPH2 R439H mice were generated on a 12956/SvEv background, as previously described (Beaulieu et al, 2008). Mice were obtained from the laboratory of Marc Caron (Duke University school of Medicine) and bred at Columbia University Medical Center. Experiments were carried out with confirmed homozygous WT and R439H littermates. Sustained-release (SR) 5-hydroxytryptophan (5-HTP SR) (≅1 g/kg/day; 6.7 mg 5-HTP per gram food) was administered to animals from =6-10 weeks in the same chow as control mice received without 5-HTP SR, as was previously done in R439H and WT mice for brain and behavior studies (Jacobsen et al, 2016b) Animal studies were approved by the IACUC of Columbia University Medical Center.

Immunocytochemistry.

Tissue samples were collected, fixed with 0.1M phosphate-buffered formaldehyde (4%; pH 7.4; from paraformaldehyde) for 1.5 h, and washed in phosphate-buffered saline (PBS). Myenteric and submucosal plexuses were examined in whole mounts of laminar preparations of the gut wall. Methods used have been described previously in our laboratory (Li et al, 2011a; Margolis et al, 2016b). Normal horse serum (10% for 30 min at room temp) was used to block preparations, which were then exposed to primary antibodies (ANNA, TH, GABA; 4° C.; 48-72 hr) that were all utilized in a prior publication (Bonnin et al, 2011). The immunoreactivities of the neuronal marker ANNA-1, tyrosine hydroxylase (TH), and γ-amino-butyric acid (GABA) were localized to quantify the abundance of total, dopaminergic and GABAergic enteric neurons (Margolis et al, 2016b). Bound primary antibodies were visualized with appropriate species-specific secondary antibodies labeled with contrasting fluorophores (Alexa Fluor™ 350, 488, or 594; diluted 1:200;). Preparations were washed (PBS), mounted in alkaline glycerol (66%; pH 8), and images were obtained with a cooled CCD camera and analyzed with computer assistance (Volocity 6.0 software, Improvision/Perkin-Elmer Life and Analytical Sciences). To count the numbers of labeled cells, a computer-controlled motorized stage was used to scan and collect images with a ×20 objective covering the entirety of a 10-mm area. Collected images were computer-processed (Volocity 6.0 software) to estimate numbers of immunoreactive cells of each type (cells per square millimeter of ganglionic area).

Colonic Propulsion.

Mice were anesthetized with isoflurane (Baxter Pharmaceutical Products) and a glass bead (3 mm in diameter) was pushed with a fire-polished glass rod through the anus into the colon to a distance of 2 cm from the anal verge (Li et al, 2006). The time required for the mice to expel the bead was determined and used to estimate in vivo colorectal propulsion.

Total gastrointestinal transit time.

Carmine red (300 μl; 6%; Sigma-Aldrich) suspended in 0.5% methylcellulose, which cannot be absorbed, was administered by gavage to study total GI transit time. Total GI transit time was considered as the interval between gavage and the appearance of carmine red in stool (Kimball et al, 2005).

Gastric Emptying and Upper Intestine Transit.

Animals were fasted overnight in cages that lacked bedding. Water was withdrawn 3 hours before the experiment. A solution containing rhodamine B dextran (100 μl; 10 mg/ml in 2% methylcellulose; Invitrogen) was administered to each mouse by gavage through a 21-gauge round-tip feeding needle. Animals were killed 15 min after gavage; the stomach, upper intestine, cecum, and colon were collected in 0.9% NaCl. The upper intestine was divided into 10 segments of equal length, and the colon (used to obtain total recovered rhodamine B fluorescence) was divided in half. Each piece of tissue was then transferred into a 14 ml tube containing 4 ml of 0.9% NaCl, homogenized, and centrifuged (2000×g) to obtain a clear supernatant. Rhodamine fluorescence was measured in 1 ml aliquots of the supernatant (VersaFluor Fluorometer; Bio-Rad Laboratories). The proportion of the rhodamine B dextran that emptied from the stomach was calculated as [(total recovered fluorescence−fluorescence remaining in the stomach)/(total recovered fluorescence)]×100. Upper intestinal transit was estimated by the position of the geometric center of the rhodamine B dextran in the upper intestine. For each segment of the upper intestine (1-10), the geometric center (a) was calculated as follows: a=(fluorescence in each segment×number of the segment)/(total fluorescence recovered in the upper intestine) (Li et al, 2011a). The total geometric center is Σ (a of each segment). Total geometric center values are distributed between 1 (minimal motility) and 10 (maximal motility).

Colonic Migrating Motor Complexes (CMMC) Patterns Measured In Vitro.

The entire colon (5-6 cm) was removed and mounted to allow spontaneous motor patterns to be video-imaged for the construction of spatiotemporal maps (Roberts et al, 2008; Roberts et al, 2007). The isolated colon was incubated in Krebs' solution until endogenous fecal pellets were expelled. The empty colon was cannulated at both ends, mounted in a horizontal organ bath, and both luminal and serosal compartments were superfused with oxygenated Krebs' solution at 35° C. The height of a reservoir connected to the oral cannula was adjusted to maintain intraluminal pressure at +2 cm HO. The anal cannula provided a maximum of 2 cm of back-pressure. The contractile activity was imaged with a Logitech Quickcam pro camera positioned 7-8 cm above the gut. Preparations were equilibrated for 30 min and four 15 min videos were captured. Spatiotemporal maps of the diameter at each point along the proximo-distal length of colon were constructed and used to quantify the frequency of CMMCs as well as the velocity and length of their propagation (Margolis et al, 2016b; Welch et al, 2014). CMMCs were defined as constrictions of the diameter of the bowel that propagated for at least 50% of the length of the preparations. For studies utilizing IR 5-HTP, a dose response curve was conducted whereby the isolated colons were exposed to concentrations of 5-HTP ranging from 1 to 10 μM, both intraluminally and extraluminally.

Quantitation of Transcripts.

Methods used to extract RNA, reverse-transcribe it to cDNA, and quantify transcript abundance with real-time PCR have been described previously (Margolis et al, 2011). Briefly, RNA was extracted from tissue with Trizol (Invitrogen, Carlsbad, Calif.) and treated with deoxyribonuclease I (1 U/mL). Polymerase chain reaction (PCR), utilizing primers for β-actin, confirmed absence of DNA contamination. Reverse transcriptase (High Capacity cDNA Archive Kit; Applied Biosystems, Foster City, Calif.) was used to convert 1 μg of sample to complementary DNA (cDNA). RT-PCR was used to quantify messenger RNA encoding TPH2, SERT, and DAT. Expression of each was normalized to that of glyceraldehyde-3-phosphate dehydrogenase (GAPDH). Primers were purchased from Applied Biosystems. The real-time reaction contained cDNA (5.0 μl), primers for the cytokine/chemokine/standard (250 nmol), PCR Master Mix (12.5 ml; Applied Biosystems), and nuclease-free water (6.25 ml). A GeneAmp 7500 sequence detection system (Applied Biosystems) was used to quantify cDNA levels. Duplicates were incubated for 2 minutes at 50° C., denatured for 10 minutes at 95° C., and subjected to 40 cycles of annealing at 60° C. for 20 seconds, extension at 60° C. for 1 minute, and denaturation at 95° C. for 15 seconds. TaqMan 7500 software (Applied Biosystems, Foster City, Calif.) was used for data analysis.

Parameters of Mucosal Maintenance.

Segments of colon and upper intestine were fixed overnight at room temperature as described above. Fixed tissue was embedded in paraffin, sectioned at 10 μm, and stained with hematoxylin and eosin. Computer-assisted imaging (Volocity 6.0 software and ImageJ 2.0 software) was employed to measure villus height and crypt perimeter (Gross et al., 2012). Villi (>30/mouse) were measured when the central lacteal was completely visualized. Villus area was quantified manually using a polygon tracing tool in ImageJ. Crypts (>30/mouse) were analyzed when the crypt-villus junction could be visualized on both sides of the crypt (Margolis et al, 2016b). Crypt perimeter was measured from the length along the edge of each crypt between two villi using a free-form line tracing tool in ImageJ.

Epithelial Staining.

Enterochromaffin cells were quantified after immunostaining with 5-HT (Immunostar, 1:1000) (Margolis et al, 2016b). Enteroendocrine cells were quantified after staining with chromogranin A (Abcam; 1:1000) (Margolis et al, 2016b). Computer-assisted imaging was used to quantify the presence of EC (5-HT+) and EE (chromogranin A+) cells.

Bound primary antibodies were visualized with appropriate species-specific secondary antibodies labeled with contrasting fluorophores (Alexa Fluor™ 350, 488, or 594; diluted 1:200).

Stool Collection for Microbiome Characterization.

5-7 pellets of fresh stool were collected after brief rectal stimulation with a cotton-tipped applicator. Pellets were immediately frozen in −80° C. until processing took place.

Microbiome Characterization.

Frozen stool specimens were thawed on ice, and 0.01-0.02 g were added to a MO BIO PowerBead Tube (MO BIO Laboratories, Carlsbad, Calif.) and vortexed for 15 minutes for gentle homogenization. Subsequent material was processed through the standard MO BIO PowerSoil extraction kit protocol (MO BIO Laboratories). Quantity and quality of the resulting nucleic acid content was confirmed by Nanodrop-1000 and Qubit (Thermo Fisher Scientific, Inc., Wilmington, Del.). Amplification and sequencing of the V4 region of the 16S ribosomal RNA gene was performed using the NEXTflex 16S V4 Amplicon-Seq Kit 2.0 (Bioo Scientific, Austin, Tex.) with 20 ng of input DNA, and sequences were generated on the Illumina MiSeq platform (Illumina, San Diego, Calif.). Sequence data were processed through the LotuS pipeline as previously described (Hildebrand et al, 2014). Briefly, reads were de-multiplexed, and paired ends were stitched. Quality filtering was performed before operational taxonomic unit (OTU) clustering using a modified version of the UPARSE algorithm (Edgar, 2013). A median of 28,476 sequences (std dev ±9388) per sample were included in the subsequent analysis. Taxonomic assignment was performed with RDP as the classifier and SILVA as the selected database (Wang et al, 2007b). Organisms potentially classified to the species level were based on individual OTUs of significance. OTUs failing to classify as bacteria at the kingdom level were removed before further analysis.

Statistical Analysis. For all studies with the exception of microbiome analysis, the student's unpaired or paired t test and 1-way ANOVA with Bonferroni correction were used, respectively, to compare single and multiple means. A P value of less than 0.05 was considered significant. For the microbiome studies, Calypso was used for the visualization and statistical analysis of OTUs, with cumulative-sum scaling and log 2 transformation performed prior to further analysis (Paulson et al, 2013; Zakrzewski et al, 2017). Principal component analysis was used to visualize overall differences between the groups. Comparisons between groups (R439H, WT, R439H+5-HTP SR, WT+5-HTP SR) were made at various taxonomic levels, including the OTU level. Taxonomic assignments for representative sequences of significant OTUs were confirmed by manual database searches and alignments. In addition, integrative analyses were conducted to evaluate in relation to neuronal counts and parameters of GI motility. Analysis of variance was performed for multiple group comparisons, and the Welch t-test was performed for comparisons between 2 groups. Pearson correlations with P-values were calculated for the OTUs and metabolites using RStudio (RStudio, Boston, Mass.). The P-values were corrected for multiple testing with the Benjamini-Hochberg method to control for false-discovery rate.

Discussion

TPH2 R441H is a SNP that is overexpressed in individuals with unipolar, severe depression (Zhang et al, 2005). TPH2 R439H mice exhibit behaviors that align broadly with the features of depression (Zhang et al, 2005), supporting the use of R439H mice as a model for analyzing potential contributions of 5-HT signaling abnormalities to depression. Bowel problems are among the most common complaints for individuals with depression (Hosseinzadeh et al, 2011). Similarly, up to one third of patients with functional constipation suffer from depression (Dipnall et al, 2016). Because TPH2 is 60-80% less effective at producing 5-HT when it harbors the R441H mutation, and because TPH2 impacts not only CNS, but also ENS development, we postulated that TPH2 hypoefficiency would not only cause abnormalities in CNS development and function but also in ENS structure and function in TPH2 R439H mice. To examine our postulate, we comprehensively analyzed the R439H mice, in which TPH2 is hypofunctional during ontogeny and throughout life, and compared them to their WT littermates.

The ENS was extremely hypoplastic in the R439H mice. Numbers of neurons were reduced in both plexuses of the upper and large intestines and neurons generated after serotonergic neurons during ontogeny (expressing TH or GABA) were more deficient than ENS neurons in general. These observations are consistent with the idea that defective 5-HT signaling due to the decreased production of 5-HT in the R439H mice interferes with enteric neurogenesis. The sensitivity of late-born neurons, particularly GABA, to TPH2 activity is consistent with the idea that serotonergic neurons, which are early-born, regulate enteric neurogenesis and thus help sculpt the ENS. These observations confirm that 5-HT is an ENS growth factor and that serotonergic signaling is essential for normal neurogenesis (Li et al, 2011a; Margolis et al, 2016b). The data are also consistent with the hypothesis that a defect common to the ENS and CNS could be responsible for comorbid GI disturbances in depression.

GI motility in the R439H mice was impaired both in vivo (slowed total GI transit time, upper intestinal transit and colonic transit) and in vitro (decreased velocity, frequency, and length of conduction of CMMCs). Because CMMCs, which are ENS-dependent (Spencer et al, 1998), were defective in isolated preparations of R439H bowel, the motor abnormality is thus an intrinsic property of the ENS. This consideration is important because this global defect in TPH2 affects the CNS and well as the ENS. The ENS hypoplasia of R439H mice thus has direct, functional consequences and could thus be a target for therapeutic intervention.

TPH2 has previously been shown to play a critical role in regulating the proliferation of crypt epithelial cells and mucosal maintenance (Gross et al, 2012). Myenteric serotonergic neurons innervate submucosal cholinergic neurons that provide a muscarinic input to the mucosa, which in turn stimulates epithelial proliferation and the growth of villi and crypts (Gross et al, 2012). Hypoefficiency of TPH2 thus decreases the action of 5-HT at submucosal synapses, which indirectly decreases villus height and crypt perimeter. Accompanying these differences were abnormalities in stem cell lineage. R439H mice exhibited a decrease in numbers of EE and EC cells. These data suggest that ENS hypoplasia due to decreased TPH2 activity during development impairs mucosal maintenance throughout life.

Since the R439H mutation results in less 5-HT production, increasing the 5-HT available for neurotransmission may ameliorate the defects in ENS development and function. Raising 5-HT levels by way of SERT antagonism, with SSRI administration, however, exaggerated the depressive behaviors and further decreased CNS 5-HT levels in the R439H mice. 5-HTP SR was found to maintain therapeutically relevant levels of CNS 5-HT in animal models of SSRI-resistant depression (Jacobsen et al, 2016a; Jacobsen et al, 2016b). We thus tested the hypothesis that 5-HTP SR could be utilized as an effective therapy to also treat the ENS abnormalities associated with TPH2 hypoefficiency. We utilized a novel sustained-release formulation of 5-HTP that, unlike immediate-release, is not acutely converted to 5-HT and thus not prematurely inactivated (Jacobsen et al, 2016a; Jacobsen et al, 2016b). Further, because 5-HTP SR does not antagonize SERT it should not exacerbate the ENS-associated defect in the R439H mice. 5-HTP SR did, in fact, overcome the R439H abnormality, reversing the ENS hypoplasia, the deficiencies of late-developing neurons, the slowing of GI transit in vivo, the defects in CMMCs that occurred in vitro as well as the abnormalities in enteric epithelial growth.

The microbiome has increasingly been associated with mood, GI motility and brain-gut axis communication. We thus evaluated the fecal microbiota to identify microbial populations that mimic those demonstrated in patients with depression and/or constipation as well as those groups that normalized following treatment with 5-HTP SR. We identified three specific microbial families in whom alterations have been found in individuals with major depressive disorder and/or constipation (Jiang et al, 2015; Lin et al, 2017) (Strati et al, 2017; Wang et al, 2017; Zhu et al, 2014; Zoppi et al, 1998) (Huang et al, 2018). These bacteria were from the Verrucomicrobia, Firmicutes, and Bacteroidetes phyla, and specifically from the Verrucomicrobiaceae, Lachnospiraceae, and Porphyromonadaceae families. These families were significantly different between the R439H and WT mice, and further, levels in the R439H mice normalized to that of WT after 5-HTP SR treatment. With regard to Verrucomicrobiaceae, Akkermansia was, in fact, totally absent in the R439H mice without 5-HTP SR. Interestingly, Akkermansia is positively correlated with colonic transit time (Vandeputte et al, 2016) such that low levels of Akkermansia muciniphila are associated with constipation as well as IBS-C and have been demonstrated to increase upon constipation treatment (Gobert et al, 2016).

Clostridiales, particularly those comprising the Lachnospiraceae family, have recently been shown to correlate with IBS-C (Gargari et al, 2018; Tap et al, 2017). Further, Lachnospiraceae has been shown to be increased in animals exposed to subchronic and mild social defeat stress, indicating that there may be some role for this family in anxiety, another trait seen in humans with the R441H mutation (Aoki-Yoshida et al, 2016). Porphyromonadaceae has been associated with a depressive-phenotype in microbiota-depleted rats (Kelly et al, 2016a). These associations may be a result of the metabolites produced by bacteria or their impact on 5-HT homeostasis. For example, Clostridia can modulate 5-HT signaling through altering SERT levels as well as the production of soluble metabolites that influence 5-HT synthesis (Reigstad et al, 2015; Yano et al, 2015) and mice that received fecal microbiota transplants from constipated individuals exhibited a reduction in intestinal peristalsis that was accompanied by alterations in levels of Clostridia, an increase in SERT expression and decrease in 5-HT content in colonic tissue (Cao et al, 2017).

Systemic administration of 5-HTP SR to the mice precludes an absolute conclusion that the actions of the drug derive from direct stimulation of enteric neurogenesis, though this seems likely and is consistent with the contributions of 5-HT to enteric neurogenesis. Regardless, the striking ability of 5-HTP SR to correct R439H-associated ENS neuronal hypoplasia and its consequences in adult mice supports the idea that TPH2 plays an important role in 5-HT-promoted neurogenesis and also supports the overall hypothesis that the defects seen in R439H mice are, in fact, due to the decreased levels of endogenous ENS-derived 5-HT produced by the hypoeffective TPH2 that these mice express.

Depression and constipation are prevalent, high-cost, high morbidity-associated medical conditions (Brody et al, 2018; Sanchez et al, 2011). Moreover, the co-occurrence of the two conditions is prevalent and significantly decreases the quality of life in those affected more than either condition in isolation (Dipnall et al, 2016; Hosseinzadeh et al, 2011). Further, pharmacological treatment of depression can often cause a worsening of GI dysfunction (Marken et al, 2000). Despite these issues, relatively little is understood about the potential factors linking the two conditions or how to treat the two conditions simultaneously in an effective manner.

This model is the first to our knowledge that possesses the face and construct validity of the behavioral, gut and enteric microbial phenotypes present in humans with constipation and depression, and allowed us to evaluate the contributions of 5-HT signaling to each of these anomalies and, further, to test a formulation of 5-HTP (SR) in the simultaneous treatment of constipation and depression. This data thus adds important insight to a growing body of literature supporting the idea that 5-HT, and specifically that a defect in 5-HT-sensitive neurogenic pathways, could underlie the behavioral and enteric abnormalities in medical conditions that affect both the brain and the intestine (Margolis et al, 2016b). A prospective human study is required to confirm that this specific mutation contributes to a co-existent depression and constipation in humans. Genetic variants in TPH2, however, have been consistently noted in human studies evaluating depressed individuals and, though scant research has been done, have begun to potentially be recognized in IBS as well, making this possibility more likely (Fasching et al, 2012; Jun et al, 2011; Karanovic et al, 2017; Tsai et al, 2009; Van der Auwera et al, 2014; Wigner et al, 2018; Zhang et al, 2005; Zill et al, 2004)

Excitingly, 5-HTP SR normalized the brain, behavioral, ENS and some microbial anomalies in our adult models. Whether 5-HTP SR might also be a novel helpful therapeutic approach for the concomitant treatment of depression and constipation in humans requires further study.

### REFERENCES

1. Brody D J, Pratt L A, Hughes J P. Prevalence of Depression Among Adults Aged 20 and Over: United States, 2013-2016. NCHS Data Brief 2018:1-8.
2. Sanchez M I, Bercik P. Epidemiology and burden of chronic constipation. Can J Gastroenterol 2011; 25 Suppl B:11B-15B.
3. Hosseinzadeh S T, Poorsaadati S, Radkani B, et al. Psychological disorders in patients with chronic constipation. Gastroenterol Hepatol Bed Bench 2011; 4:159-63.
4. Dipnall J F, Pasco J A, Berk M, et al. Into the Bowels of Depression: Unravelling Medical Symptoms Associated with Depression by Applying Machine-Learning Techniques to a Community Based Population Sample. PLoS One 2016; 11:e0167055.
5. Rao M, Gershon M D. The bowel and beyond: the enteric nervous system in neurological disorders. Nat Rev Gastroenterol Hepatol 2016; 13:517-28.
6. Gershon M D. 5-Hydroxytryptamine (serotonin) in the gastrointestinal tract. Current opinion in endocrinology, diabetes, and obesity 2013; 20:14-21.
7. Furness J B. The enteric nervous system: normal functions and enteric neuropathies. Neurogastroenterol Motil 2008; 20 Suppl 1:32-8.
8. Erspamer V. Occurrence of indolealkylamines in nature. In: Erspamer V, ed. Handbook of Experimental Pharmacology: 5-Hydroxytryptamine and Related Indolealkylamines. Volume 19. New York: Springer-Verlag, 1966:132-181.
9. Erspamer V. Uber den 5-Hydroytryptamin-(Enteramin) Gehalt des Magen-Darmtraktes bei den Wirbelten. Naturwissenschaften 1953; 40:318-319.
10. Matthes S, Bader M. Peripheral Serotonin Synthesis as a New Drug Target. Trends Pharmacol Sci 2018; 39:560-572.
11. Gershon M D, Tack J. The serotonin signaling system: from basic understanding to drug development for functional GI disorders. Gastroenterology 2007; 132:397-414.
12. Gregory R E, Ettinger D S. 5-HT₃ receptor antagonists for the prevention of chemotherapy-induced nausea and vomiting. A comparison of their pharmacology and clinical efficacy. Drugs 1998; 55.
13. Hagbom M, Istrate C, Engblom D, et al. Rotavirus stimulates release of serotonin (5-HT) from human enterochromaffin cells and activates brain structures involved in nausea and vomiting. PLoS Pathog 2011; 7:e1002115.
14. Blackshaw L A, Grundy D. Effects of 5-hydroxytryptamine on discharge of vagal mucosal afferent fibres from the upper gastrointestinal tract of the ferret. J. Auton. Nerv. Syst. 1993; 45:41-50.
15. Grundy D, Blackshaw L A, Hillsley K. Role of 5-hydroxytryptamine in gastrointestinal chemosensitivity. Dig. Dis. Sci. 1994; 39 (12 Suppl):44S-47S.
16. Hillsley K, Kirkup A J, Grundy D. Direct and indirect actions of 5-hydroxytryptamine on the discharge of mesenteric afferent fibers innervating the rat jejunum. J. Physiol. (Lond.) 1998; 506:551-561.
17. Liu M T, Kuan Y H, Wang J, et al. 5-HT4 receptor-mediated neuroprotection and neurogenesis in the enteric nervous system of adult mice. J Neurosci 2009; 29:9683-99.
18. Margolis K G, Li Z S, Stevanovic K, et al. Serotonin transporter variant drives preventable gastrointestinal abnormalities in development and function. Journal of Clinical Investigation 2016; 126:2221-2235.
19. Li Z, Chalazonitis A, Huang Y Y, et al. Essential roles of enteric neuronal serotonin in gastrointestinal motility and the development/survival of enteric dopaminergic neurons. J. Neurosci. 2011; 31:8998-9009.
20. Margolis K G, Li Z, Stevanovic K, et al. Serotonin transporter variant drives preventable gastrointestinal abnormalities in development and function. J Clin Invest 2016; 126:2221-35.
21. Gross E R, Gershon M D, Margolis K G, et al. Neuronal serotonin regulates growth of the intestinal mucosa in mice. Gastroenterology 2012; 143:408-17 e2.
22. Margolis K G, Gershon M D. Enteric Neuronal Regulation of Intestinal Inflammation. Trends Neurosci 2016; 39:614-624.
23. Li Z, Chalazonitis A, Huang Y Y, et al. Essential roles of enteric neuronal serotonin in gastrointestinal motility and the development/survival of enteric dopaminergic neurons. J Neurosci 2011; 31:8998-9009.
24. Heredia D J, Gershon M D, Koh S D, et al. Important role of mucosal serotonin in colonic propulsion and peristaltic reflexes: in vitro analyses in mice lacking tryptophan hydroxylase 1. J Physiol 2013; 591:5939-57.
25. Smith T K, Gershon M D. CrossTalk proposal: 5-HT is necessary for peristalsis. J Physiol 2015; 593:3225-7.
26. Gaspar P, Cases O, Maroteaux L. The developmental role of serotonin: news from mouse molecular genetics. Nat Rev Neurosci 2003; 4:1002-12.
27. Cowen P J, Browning M. What has serotonin to do with depression? World Psychiatry 2015; 14:158-60.
28. Kendig D M, Grider J R. Serotonin and colonic motility. Neurogastroenterol Motil 2015; 27:899-905.
29. Walther D J, Peter J U, Bashammakh S, et al. Synthesis of serotonin by a second tryptophan hydroxylase isoform. Science 2003; 299:76.
30. Stahl S M. Mechanism of action of serotonin selective reuptake inhibitors. Serotonin receptors and pathways mediate therapeutic effects and side effects. J Affect Disord 1998; 51:215-35.
31. Willner P, Scheel-Kruger J, Belzung C. The neurobiology of depression and antidepressant action. Neurosci Biobehav Rev 2013; 37:2331-71.
32. Jacobsen J P R, Krystal A D, Krishnan K R R, et al. Adjunctive 5-Hydroxytryptophan Slow-Release for Treatment-Resistant Depression: Clinical and Preclinical Rationale. Trends Pharmacol Sci 2016; 37:933-944.
33. Trivedi M H, Rush A J, Wisniewski S R, et al. Evaluation of outcomes with citalopram for depression using measurement-based care in STAR*D: implications for clinical practice. Am J Psychiatry 2006; 163:28-40.
34. Marken P A, Munro J S. Selecting a Selective Serotonin Reuptake Inhibitor: Clinically Important Distinguishing Features. Prim Care Companion J Clin Psychiatry 2000; 2:205-210.
35. Wigner P, Czarny P, Synowiec E, et al. Association between single nucleotide polymorphisms of TPH1 and TPH2 genes, and depressive disorders. J Cell Mol Med 2018; 22:1778-1791.
36. Karanovic J, Ivkovic M, Jovanovic V M, et al. Effect of childhood general traumas on suicide attempt depends on TPH2 and ADARB1 variants in psychiatric patients. J Neural Transm (Vienna) 2017; 124:621-629.
37. Van der Auwera S, Janowitz D, Schulz A, et al. Interaction among childhood trauma and functional polymorphisms in the serotonin pathway moderate the risk of depressive disorders.

Eur Arch Psychiatry Clin Neurosci 2014; 264 Suppl 1:S45-54.
38. Fasching P A, Faschingbauer F, Goecke T W, et al. Genetic variants in the tryptophan hydroxylase 2 gene (TPH2) and depression during and after pregnancy. J Psychiatr Res 2012; 46:1109-17.
39. Tsai S J, Hong C J, Liou Y J, et al. Tryptophan hydroxylase 2 gene is associated with major depression and antidepressant treatment response. Prog Neuropsychopharmacol Biol Psychiatry 2009; 33:637-41.
40. Zill P, Baghai T C, Zwanzger P, et al. SNP and haplotype analysis of a novel tryptophan hydroxylase isoform (TPH2) gene provide evidence for association with major depression. Mol Psychiatry 2004; 9:1030-6.
41. Zhang X, Gainetdinov R R, Beaulieu J M, et al. Loss-of-function mutation in tryptophan hydroxylase-2 identified in unipolar major depression. Neuron 2005; 45:11-6.
42. Sachs B D, Jacobsen J P, Thomas T L, et al. The effects of congenital brain serotonin deficiency on responses to chronic fluoxetine. Transl Psychiatry 2013; 3:e291.
43. Sachs B D, Rodriguiz R M, Siesser W B, et al. The effects of brain serotonin deficiency on behavioural disinhibition and anxiety-like behaviour following mild early life stress. Int J Neuropsychopharmacol 2013; 16:2081-94.
44. Siesser W B, Sachs B D, Ramsey A J, et al. Chronic SSRI treatment exacerbates serotonin deficiency in humanized Tph2 mutant mice. ACS Chem Neurosci 2013; 4:84-8.
45. Jacobsen J P, Medvedev T O, Caron M G. The 5-HT deficiency theory of depression: perspectives from a naturalistic 5-HT deficiency model, the tryptophan hydroxylase 2Arg439His knockin mouse. Philos Trans R Soc Lond B Biol Sci 2012; 367:2444-59.
46. Jacobsen J P, Siesser W B, Sachs B D, et al. Deficient serotonin neurotransmission and depression-like serotonin biomarker alterations in tryptophan hydroxylase 2 (Tph2) loss-of-function mice. Mol Psychiatry 2012; 17:694-704.
47. Burokas A, Moloney R D, Dinan T G, et al. Microbiota regulation of the Mammalian gut-brain axis. Adv Appl Microbiol 2015; 91:1-62.
48. Dinan T G, Cryan J F. Gut-brain axis in 2016: Brain-gut-microbiota axis—mood, metabolism and behaviour. Nat Rev Gastroenterol Hepatol 2017; 14:69-70.
49. Kelly J R, Clarke G, Cryan J F, et al. Brain-gut-microbiota axis: challenges for translation in psychiatry. Ann Epidemiol 2016; 26:366-72.
50. Yano J M, Yu K, Donaldson G P, et al. Indigenous bacteria from the gut microbiota regulate host serotonin biosynthesis. Cell 2015; 161:264-76.
51. Obata Y, Pachnis V. The Effect of Microbiota and the Immune System on the Development and Organization of the Enteric Nervous System. Gastroenterology 2016; 151:836-844.
52. Sampson T R, Mazmanian S K. Control of brain development, function, and behavior by the microbiome. Cell Host Microbe 2015; 17:565-76.
53. O'Mahony S M, Clarke G, Bone Y E, et al. Serotonin, tryptophan metabolism and the brain-gut-microbiome axis. Behav Brain Res 2015; 277:32-48.
54. Reigstad C S, Salmonson C E, Rainey J F, 3rd, et al. Gut microbes promote colonic serotonin production through an effect of short-chain fatty acids on enterochromaffin cells.

FASEB J 2015; 29:1395-403.
55. Quigley E M, Spiller R C. Constipation and the Microbiome: Lumen Versus Mucosa! Gastroenterology 2016; 150:300-3.
56. Tigchelaar E F, Bonder M J, Jankipersadsing S A, et al. Gut microbiota composition associated with stool consistency. Gut 2016; 65:540-2.
57. Kelly J R, Bone Y, C O B, et al. Transferring the blues: Depression-associated gut microbiota induces neurobehavioural changes in the rat. J Psychiatr Res 2016; 82:109-18.
58. Yarandi S S, Peterson D A, Treisman G J, et al. Modulatory Effects of Gut Microbiota on the Central Nervous System: How Gut Could Play a Role in Neuropsychiatric Health and Diseases. J Neurogastroenterol Motil 2016; 22:201-12.
59. Jiang H, Ling Z, Zhang Y, et al. Altered fecal microbiota composition in patients with major depressive disorder. Brain Behav Immun 2015; 48:186-94.
60. Lin P, Ding B, Feng C, et al. Prevotella and Klebsiella proportions in fecal microbial communities are potential characteristic parameters for patients with major depressive disorder. J Affect Disord 2017; 207:300-304.
61. Zoppi G, Cinquetti M, Luciano A, et al. The intestinal ecosystem in chronic functional constipation. Acta Paediatr 1998; 87:836-41.
62. Wang L, Hu L, Xu Q, et al. Bifidobacterium adolescentis Exerts Strain-Specific Effects on Constipation Induced by Loperamide in BALB/c Mice. Int J Mol Sci 2017; 18.
63. Strati F, Cavalieri D, Albanese D, et al. New evidences on the altered gut microbiota in autism spectrum disorders. Microbiome 2017; 5:24.
64. Zhu L, Liu W, Alkhouri R, et al. Structural changes in the gut microbiome of constipated patients. Physiol Genomics 2014; 46:679-86.
65. Huang L S, Kong C, Gao R Y, et al. Analysis of fecal microbiota in patients with functional constipation undergoing treatment with synbiotics. Eur J Clin Microbiol Infect Dis 2018; 37:555-563.
66. Wikoff W R, Anfora A T, Liu J, et al. Metabolomics analysis reveals large effects of gut microflora on mammalian blood metabolites. Proc Natl Acad Sci USA 2009; 106:3698-703.
67. Oleskin A V, Kirovskaia T A, Botvinko I V, et al. [Effect of serotonin (5-hydroxytryptamine) on the growth and differentiation of microorganisms]. Mikrobiologiia 1998; 67:305-12.
68. Tsavkelova E A, Klimova S, Cherdyntseva T A, et al. [Hormones and hormone-like substances of microorganisms: a review]. Prikl Biokhim Mikrobiol 2006; 42:261-8.
69. Jacobsen J P, Rudder M L, Roberts W, et al. SSRI Augmentation by 5-Hydroxytryptophan Slow Release: Mouse Pharmacodynamic Proof of Concept. Neuropsychopharmacology 2016; 41:2324-34.
70. Nardini M, De Stefano R, Iannuccelli M, et al. Treatment of depression with L-5-hydroxytryptophan combined with chlorimipramine, a double-blind study. Int J Clin Pharmacol Res 1983; 3:239-50.
71. Karaosmanoglu T, Aygun B, Wade P R, et al. Regional differences in the number of neurons in the myenteric plexus of the guinea pig small intestine and colon: an evaluation of markers used to count neurons. Anat Rec 1996; 244:470-80.
72. Spencer N J, Bywater R A, Taylor G S. Evidence that myoelectric complexes in the isolated mouse colon may not be of myogenic origin Neurosci Lett 1998; 250:153-6.
73. Wang L, Martinez V, Kimura H, et al. 5-Hydroxytryptophan activates colonic myenteric neurons and propulsive motor function through 5-HT4 receptors in conscious mice. Am J Physiol Gastrointest Liver Physiol 2007; 292:G419-28.
74. Schemann M, Ehrlein H J. 5-Hydroxytryptophan and cisapride stimulate propulsive jejunal motility and transit of chyme in dogs. Digestion 1986; 34:229-35.
75. Bueno L, Fioramonti J. A possible central serotonergic mechanism involved in the effects of morphine on colonic motility in dog. Eur J Pharmacol 1982; 82:147-53.
76. Gorard D A, Libby G W, Farthing M J. 5-Hydroxytryptamine and human small intestinal motility: effect of inhibiting 5-hydroxytryptamine reuptake. Gut 1994; 35:496-500.
77. Bogdanski D F, Weissbach H, Udenfriend. Pharmacological studies with the serotonin precursor, 5-hydroxytryptophan. J Pharmacol Exp Ther 1958; 122:182-94.
78. Beaulieu J M, Zhang X, Rodriguiz R M, et al. Role of GSK3 beta in behavioral abnormalities induced by serotonin deficiency. Proc Natl Acad Sci USA 2008; 105:1333-8.
79. Bonnin A, Goeden N, Chen K, et al. A transient placental source of serotonin for the fetal forebrain. Nature 2011; 472:347-50.
80. Li Z S, Schmauss C, Cuenca A, et al. Physiological modulation of intestinal motility by enteric dopaminergic neurons and the D2 receptor: analysis of dopamine receptor expression, location, development, and function in wild-type and knock-out mice. J Neurosci 2006; 26:2798-807.
81. Kimball E S, Palmer J M, D'Andrea M R, et al. Acute colitis induction by oil of mustard results in later development of an IBS-like accelerated upper GI transit in mice. Am J Physiol Gastrointest Liver Physiol 2005; 288:G1266-73.
82. Roberts R R, Bornstein J C, Bergner A J, et al. Disturbances of colonic motility in mouse models of Hirschsprung's disease. Am J Physiol Gastrointest Liver Physiol 2008; 294:G996-G1008.
83. Roberts R R, Murphy J F, Young H M, et al. Development of colonic motility in the neonatal mouse-studies using spatiotemporal maps. American journal of physiology. Gastrointestinal and liver physiology 2007; 292:G930-8.
84. Welch M G, Margolis K G, Li Z, et al. Oxytocin regulates gastrointestinal motility, inflammation, macromolecular permeability, and mucosal maintenance in mice. Am J Physiol Gastrointest Liver Physiol 2014; 307:G848-62.
85. Margolis K G, Stevanovic K, Karamooz N, et al. Enteric neuronal density contributes to the severity of intestinal inflammation. Gastroenterology 2011; 141:588-98, 598 e1-2.
86. Hildebrand F, Tadeo R, Voigt A Y, et al. LotuS: an efficient and user-friendly OTU processing pipeline. Microbiome 2014; 2:30.
87. Edgar R C. UPARSE: highly accurate OTU sequences from microbial amplicon reads. Nat Methods 2013; 10:996-8.
88. Wang Q, Garrity G M, Tiedje J M, et al. Naive Bayesian classifier for rapid assignment of rRNA sequences into the new bacterial taxonomy. Appl Environ Microbiol 2007; 73:5261-7.
89. Zakrzewski M, Proietti C, Ellis J J, et al. Calypso: a user-friendly web-server for mining and visualizing microbiome-environment interactions. Bioinformatics 2017; 33:782-783.
90. Paulson J N, Stine O C, Bravo H C, et al. Differential abundance analysis for microbial marker-gene surveys. Nat Methods 2013; 10:1200-2.
91. Vandeputte D, Falony G, Vieira-Silva S, et al. Stool consistency is strongly associated with gut microbiota richness and composition, enterotypes and bacterial growth rates. Gut 2016; 65:57-62.
92. Gobert A P, Sagrestani G, Delmas E, et al. The human intestinal microbiota of constipated-predominant irritable bowel syndrome patients exhibits anti-inflammatory properties. Sci Rep 2016; 6:39399.
93. Gargari G, Tavemiti V, Gardana C, et al. Fecal Clostridiales distribution and short-chain fatty acids reflect bowel habits in irritable bowel syndrome. Environ Microbiol 2018.
94. Tap J, Derrien M, Tornblom H, et al. Identification of an Intestinal Microbiota Signature Associated With Severity of Irritable Bowel Syndrome. Gastroenterology 2017; 152:111-123 e8.
95. Aoki-Yoshida A, Aoki R, Moriya N, et al. Omics Studies of the Murine Intestinal Ecosystem Exposed to Subchronic and Mild Social Defeat Stress. J Proteome Res 2016; 15:3126-38.
96. Cao H, Liu X, An Y, et al. Dysbiosis contributes to chronic constipation development via regulation of serotonin transporter in the intestine. Sci Rep 2017; 7:10322.
97. Jun S, Kohen R, Cain K C, et al. Associations of tryptophan hydroxylase gene polymorphisms with irritable bowel syndrome. Neurogastroenterol Motil 2011; 23:233-9, e116.

### Example 2: Human 5-HTP Gastrointestinal Regional Absorption Study

Multiple previous clinical studies report that 5-HTP is readily absorbed by upper intestine, to an extent sufficient to cause pharmacologically active systemic 5-HTP levels (Lowe et al, 2006; Meltzer et al, 1984; Sargent et al, 1998). No published clinical study reports on 5-HTP's absorption by the colon. However, one rodent study reported that 5-HTP was absorbed equally well by the colon and the upper intestine (Jacobsen et al, 2016a).

Human gastrointestinal absorption of 5-HTP was tested in the following study.

Methods

Determination of absolute and regional intestinal bioavailability of 5-HTP in humans.

5-HTP dosing:

The free base form of 5-HTP was used (5-HTP has a water solubility of >10 mg/mL).

Colonic: 5-HTP free base saline solution 200 mg.

Intravenous: 5-HTP free base saline solution 50 mg.

Oral/Upper GI: Two 5-HTP gelatin tablets of 100 mg 5-HTP free base (200 mg total dose).

Subjects:

Healthy male and female volunteers aged 18 to 65 years with a body mass index (BMI) of 19 to 28 were eligible for the study. Subjects were admitted to the investigational medical unit (IMU) 2 h before 5-HTP administration and remained at the IMU for 24 h following, for blood sampling and safety assessment.

Study Sequence:

All subjects (n=12) received 5-HTP mg on 3 occasions. (1) Intra-colonic 200 mg 5-HTP solution by colonoscopy. (2) Intravenous 50 mg (IV). (3) Upper GI (oral) 5-HTP 200 mg. At least 6 days passed between each visit.

Plasma Samples Analysis:

Plasma samples were collected over 24-hours after 5-HTP administration. Plasma samples were stored at −80° C. until analysis. 5-HTP and the metabolite 5-hydroxyindole-acetic-acid (5-HIAA) were quantified by liquid chromatography with mass-spectrometry detection.

Data Analysis:

The PK data were analyzed using noncompartmental (NCA) and compartmental (mixed effects) mathematical modelling approaches, to calculate area 5-HTP plasma under the curve (AUC) for each subject for each 5-HTP administration. This data was used for calculating the 5-HTP absolute bioavailability (F) and relative upper GI tract:colon bioavailability (RBA) data, according to formulas provided below.

Results

The human bioavailability of 5-HTP via the various routes of administration above was established in human subjects by administering 5-HTP through these various routes and quantifying the resultant 5-HTP plasma levels at various time points. All human subjects received 5-HTP via each of the three administration routes on separate days. Plasma samples for 5-HTP quantification were collected for 24-hour at selected time points and the results are shown in FIG. 10.

From FIG. 10, the area under the curve (AUC) for each route of administration was obtained, which was then used to calculate the absolute bioavailability (F) of the oral and colon routes of administration. For example, the formula for calculating F for a drug administered by the oral route (po) is given below (D is dose):

$${F}_{po} = 100 \% \times \frac{{AUC}_{po} \times {D}_{iv}}{{AUC}_{iv} \times {D}_{po}}$$

A similar formula was used to calculate the absolute bioavailability after colonic dosing. The AUC for oral dosing was 1505 (h*ng/ml), for colonic dosing it was 312 (h*ng/ml), and for intravenous dosing it was 2042 (h*ng/ml), which values were used to provide oral and colonic bioavailabilities, as shown below.

$${F}_{po} = 100 \% \times \frac{1505 ( h * ng \text{/} ml ) \times 50 mg}{2042 ( h * ng \text{/} ml ) \times 200 mg} \approx 20 \%$$

$${F}_{colon} = 100 \% \times \frac{312 ( h * ng \text{/} ml ) \times 50 mg}{2042 ( h * ng \text{/} ml ) \times 200 mg} \approx 4 \%$$

Based on the calculations above, the absolute 5-HTP bioavailability through the oral route was F=20% (upper GI tract), while the absolute bioavailability of 5-HTP from colonic dosing was F=4%. The relative bioavailability oral:colon (RBA) was calculated as follows:

$${F}_{rel} = 100 \% \times \frac{{AUC}_{co} \times {D}_{po}}{{AUC}_{po} \times {D}_{co}}$$

Thus:

$${F}_{rel} = 100 \% \times \frac{312 ( h * ng \text{/} ml ) \times 200 mg}{1505 ( h * ng \text{/} ml ) \times 200 mg} \approx 20 \%$$

As calculated above, the relative upper GI tract:colon bioavailability was 20% (corresponding to an absolute colonic bioavailability of 4%).

Discussion

These data demonstrate that 5-HTP delivered to the colon is minimally absorbed into the systemic circulation and will cause at most minimal increases of systemic 5-HTP and hence minimal increases in 5-HT synthesis in non-GI peripheral organs and in the brain. Indeed, the maximal 5-HTP plasma levels observed after colonic administration of 5-HTP 200 mg, ˜40-50 ng/ml, was still within the range observed at baseline in untreated humans (Comai et al, 2010). This contrasts to when 5-HTP is delivered to the upper intestine, where 5-HTP is absorbed with 5 times higher efficacy compared to in the colon, which causes substantial increase in systemic 5-HTP, which in turn is well-known from the art to increase 5-HT synthesis in non-GI peripheral organs and the brain.

Therefore, 5-HTP delivery selectively to the colon may eliminate or minimize the probability of systemic adverse effects from 5-HTP-induced 5-HT synthesis, e.g. nausea, vomiting, stomach pain, sedation, and dizziness. Further, 5-HTP delivery selectively to the colon may eliminate or minimize the probability of 5-HTP interacting with other serotonergic medications, e.g. antidepressant and triptans. Moreover, 5-HTP delivery to the colon will concentrate 5-HTP to a primary site of pathology in constipation.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

1. A method of treating or ameliorating the effects of a gastrointestinal (GI) condition in a subject in need thereof, comprising administering to the subject an effective amount of a sustained release formulation of 5-hydroxytryptophan (5-HTP SR).

2. The method according to claim 1, wherein the GI condition is constipation.

3. The method according to claim 1, wherein the subject is selected from the group consisting of humans, veterinary animals, and agricultural animals.

4. The method according to claim 1, wherein the subject is a human.

5. The method according to claim 1, wherein the gastrointestinal (GI) condition is selected from idiopathic constipation, constipation-predominant Irritable Bowel Syndrome (IBS-C), opioid-induced constipation, constipation in Parkinson's and constipation in autism.

6. The method according to claim 1, wherein the gastrointestinal (GI) condition is idiopathic constipation or constipation-predominant Irritable Bowel Syndrome (IBS-C).

7. The method according to claim 1, wherein the gastrointestinal (GI) condition is constipation induced by a drug treatment (e.g., opioid treatment).

8. The method according to claim 1, wherein the subject has GI 5-HT deficiency.

9. The method according to claim 1, wherein the subject has a mutation in a gene encoding tryptophan hydroxylase 2 (TPH2).

10. The method according to claim 9, wherein the mutation is a R441H or equivalent mutation in the gene.

11. The method according to claim 1, wherein the administering step is carried out by oral administration.

12. The method according to claim 1, wherein the sustained release formulation of 5-hydroxytryptophan (5-HTP SR) is administered at a rate of about 0.1 g per kg body weight per day.

13. The method according to claim 1, wherein the sustained release formulation of 5-hydroxytryptophan (5-HTP SR) is administered at a rate of about 0.01 to about 0.1 g per kg body weight per day, or about 0.001 to about 0.01 g per kg body weight per day, or about 0.0001 to about 0.001 g per kg body weight per day, or about 0.00001 to about 0.0001 g per kg body weight per day.

14. The method according to claim 1, wherein the sustained release formulation of 5-hydroxytryptophan (5-HTP SR) is orally administered at a dosage of from 5, 10 or 25 mg to 100, 250 or 500 mg; or orally administered at a dosage of from 0.5 to 1 gram.

15. A method for treating visceral pain in a subject in need thereof, comprising administering to the subject an effective amount of a sustained release formulation of 5-hydroxytryptophan (5-HTP SR).

16. A kit for treating or ameliorating the effects of a gastrointestinal (GI) condition in a subject, comprising an effective amount of a sustained release formulation of 5-hydroxytryptophan (5-HTP SR), together with a second active compound, and optionally instructions for its use.

17. The kit of claim 16, wherein the second active compound is a pro-secretory compound.

18. The kit of claim 16, wherein the second active compound is an osmotic laxative.

19. The kit of claim 16, wherein the second active compound is a stimulant laxative.

20. The method according to claim 1, where the 5-HTP SR is a gastroretentive formulation optionally comprising a peripheral decarboxylase inhibitor (e.g., at a low dose not causing systemically pharmacologically active blood levels (e.g., in the range of 5-50 mg per day, or 1-10 mg, 2-20 mg, or 20-50 mg per day ranges)).

21. The method according to claim 1, where 5-HTP is delivered to the upper and lower GI, or where 5-HTP is delivered to the intestine, excluding the stomach.

22. The method according to claim 1, where 5-HTP is delivered selectively to the colon.

23.-25. (canceled)
